SEQUENCE LISTING (25 sequences)

SEQ ID NO: 1 - protein, 9 aa, Artificial Sequence
  Synthetic Peptide
YFPGQFAFS

SEQ ID NO: 2 - protein, 25 aa, Artificial Sequence
  Synthetic Peptide
MDNYTVAPDDEYDVLILDDYLDNSC

SEQ ID NO: 3 - DNA, 20 bp, Artificial Sequence
  Synthetic Primer
ttccaacatcctcctccttg

SEQ ID NO: 4 - DNA, 20 bp, Artificial Sequence
  Synthetic Primer
gatgcacgcaacaataccac

SEQ ID NO: 5 - DNA, 23 bp, Artificial Sequence
  Synthetic Primer
gagctgtttgcagaccaaagttc

SEQ ID NO: 6 - DNA, 20 bp, Artificial Sequence
  Synthetic Primer
ccctggcacatgaatcctgg

SEQ ID NO: 7 - protein, 18 aa, Artificial Sequence
  Synthetic Peptide
ADPELTEFAPHHHHHHHH

SEQ ID NO: 8 - protein, 18 aa, Artificial Sequence
  Synthetic Peptide
ADPELTELPRSPHHHHHH

SEQ ID NO: 9 - protein, 18 aa, Artificial Sequence
  Synthetic Peptide
ADPTEPEFAPHHHHHHHH

SEQ ID NO: 10 - protein, 11 aa, Artificial Sequence
  Synthetic Peptide
ADPELTEFAPH

SEQ ID NO: 11 - protein, 11 aa, Artificial Sequence
  Synthetic Peptide
ADPELTERSPH

SEQ ID NO: 12 - protein, 11 aa, Artificial Sequence
  Synthetic Peptide
ADPTEPEFAPH

SEQ ID NO: 13 - protein, 9 aa, Artificial Sequence
  Synthetic Peptide
YFPGQFAFS

SEQ ID NO: 14 - DNA, 16 bp, Homo sapiens
tggtttcacttttgca

SEQ ID NO: 15 - DNA, 16 bp, Pan troglodytes
tggtttcacttttgca

SEQ ID NO: 16 - DNA, 16 bp, Mus musculus
cagtttcacttttgca

SEQ ID NO: 17 - DNA, 16 bp, Rattus sp.
cagtttcacttttgca

SEQ ID NO: 18 - DNA, 16 bp, Canis lupus
tggtttcacttttgca

SEQ ID NO: 19 - DNA, 7 bp, Homo sapiens
attatac

SEQ ID NO: 20 - DNA, 8 bp, Pan troglodytes
gtttatac

SEQ ID NO: 21 - DNA, 8 bp, Mus musculus
gtttatat

SEQ ID NO: 22 - DNA, 8 bp, Rattus sp.
gtttatat

SEQ ID NO: 23 - DNA, 8 bp, Canis lupus
gtttctgt

SEQ ID NO: 24 - protein, 39 aa, Homo sapiens
MANYTLAPEDEYDVLIEGELESDEAEQCDKYDAQALSAQ

SEQ ID NO: 25 - protein, 40 aa, Mus musculus
MDNYTVAPDDEYDVLILDDYLDNSGPDQVPAPEFLSPQQV

(19) United States
(12) Patent Application Publication
(10) Pub. No.: US 2010/0092974 A1
(43) Pub. Date: Apr. 15, 2010

(54) METHODS FOR SCREENING FOR MODULATORS OF CCRL2

(75) Inventors: Brian A. Zabel, Redwood City, CA (US); Eugene C. Butcher, Portola Valley, CA (US); Stephen J. Galli, Stanford, CA (US); Susumu Nakae, Machida (JP); Luis Zuniga, Palo Alto, CA (US)

Correspondence Address: Stanford University Office of Technology Licensing;Bozicevic, Field & Francis LLP, 1900 University Avenue, Suite 200, East Palo Alto, CA, 94303, US

(73) Assignee: The Board of Trustees of the Leland Stanford Junior University

(21) Appl. No.: 12/541,056

(22) Filed: Aug. 13, 2009

Related U.S. Application Data
(60) Provisional application No. 61/089,416, filed on Aug. 15, 2008.

(51) Int. Cl. C12Q 1/68

(52) U.S. Cl. 435/6

(57) ABSTRACT

The invention provides methods and compositions for identifying a modulator of CCRL2 and chemerin. The present invention also provides methods and compositions for treating an inflammatory disease by administering a compound that modulates the interaction of CCRL2 with chemerin.

### CROSS-REFERENCE

This application claims the benefit of U.S. Provisional Application No. 61/089,416, filed Aug. 15, 2008, which application is incorporated by reference herein in its entirety.

### GOVERNMENT RIGHTS

This invention was made with Government support under federal grants DK056339, AI056339, AI059635, AI07290, AI047822, GM037734, AI057229, AI070813, AI023990, CA072074, and AI079320 awarded by the National Institutes of Health and with support from the Department of Veteran's Affairs. The Government has certain rights in this invention.

### BACKGROUND OF THE INVENTION

Chemoattractants acting through their cognate receptors are critical for the recruitment of effector immune cells to inflamed tissues, and are therefore of considerable interest as potential targets for the treatment of inflammatory disease. CCRL2 (also known as HCR, CRAM-A and CRAM-B) encodes an orphan chemokine receptor-like protein, which is predicted to be a seven transmembrane protein. G protein coupled receptors (GPCRs) are a family of approximately 500 proteins with a 7 transmembrane structure that are involved in variety of biological functions.

For classical chemoattractant receptors, interaction with its cognate ligand causes a conformational change in the protein and facilitates the binding of small associated heterotrimeric G proteins to the intracellular receptor domains, which initiate a signaling cascade. ‘Atypical’ chemoattractant receptors bind to chemoattractants but do not transduce intracellular signals leading to cell migration. This functionally defined receptor subfamily is currently comprised of three members—D6, DARC (Duffy antigen receptor for chemokines), and CCX-CKR (Chemocentryx chemokine receptor) (Comerford, I., W. Litchfield, Y. Harata-Lee, R. J. Nibbs, and S. R. McColl. 2007. Bioessays 29: 237-247; Mantovani, A., R. Bonecchi, and M. Locati. 2006. Nat Rev Immunol 6: 907-918). The receptors are also referred to as professional chemokine “interceptors”, a name that reflects their ability to efficiently internalize bound ligand (Haraldsen, G., and A. Rot. 2006. Eur J Immunol 36: 1659-1661). GPCRs are cell surface receptors and therefore are attractive targets for pharmacological intervention. CCRL2 has been shown to be expressed at high levels in primary neutrophils and primary monocytes, and is further upregulated on neutrophil activation and when monocytes differentiate to macrophages.

### SUMMARY OF THE INVENTION

The invention provides methods and compositions for identifying a modulator of CCRL2 and chemerin. The present invention also provides methods and compositions for treating an inflammatory disease by administering a compound that modulates the interaction of CCRL2 with chemerin.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Mast cell expression of mCCRL2. Panel A: generation of anti-mCCRL2 specific mAbs. Unlabeled mCCRL2/L1.2 transfectants were mixed 1:1 with CMFDA-labeled CCR10/L1.2 transfectants and used to identify mCCRL2-specific mAbs by flow cytometry. Panel B: freshly isolated peritoneal leukocytes were harvested and mCCRL2 expression was evaluated on SSC^{high} F4/80⁻ c-kit⁺ mast cells. Panel C: F4/80⁻ mCCRL2⁺ and F4/80⁻ c-kit⁺ peritoneal cells were sorted, harvested by cytospin, and stained by Wright-Giemsa. Cells were examined by light microscope using a 40× objective; scale bars=10 μm. Panel D: bone marrow-derived cultured mast cells (BMCMCs) were generated and stained for mCCRL2 reactivity. Panel E: the relative RNA expression of mCCRL2 was assessed in mast cells by real time quantitative PCR. The expression data were normalized to Cyclophilin A and displayed relative to mCCRL2 expression in the spleen (set=1.0). Each bar represents the mean±SD of triplicate wells; ND, not detectable; RT, reverse transcriptase. One representative data set of the at least 3 experiments, each of which gave similar results, is shown for each part of this figure.

FIG. 2. mCCRL2 KO mice. Panel A: mCCRL2 KO mice are deficient in CCRL2 protein expression. Freshly isolated peritoneal leukocytes were harvested and mCCRL2 expression was evaluated on SSC^{high} F4/80-ckit+ mast cells. A representative histogram plot of the at least 3 independent experiments performed, each of which gave similar results, is shown. Panel B: enumeration of ear skin and mesenteric mast cells in WT and KO mice. Ear skin: KO (n=9), WT, het (n=4,1), 4 sections/mouse. Mesenteric window: KO (n=18), WT, het (n=9,2).

FIG. 3. BMCMCs from mCCRL2 KO and WT mice display similar functional responses in vitro. Panel A: in vitro transwell chemotaxis to stem cell factor (SCF). Four populations of BMCMCs were tested, with duplicate wells for each genotype. The mean±SEM is displayed. Panels B-D: BMCMCs were sensitized with DNP-specific IgE and then activated by addition of DNP-HSA. The following parameters were measured: (Panel B) Degranulation (as quantified by β-hexoaminidase release), (Panel C) TNFα and IL-6 secretion, and (Panel D) Upregulation of co-stimulatory molecules CD137 and CD153. Panel E: BMCMC-stimulated T cell proliferation. Naive T cells were incubated as indicated with anti-CD3, and co-cultured with mitomycin C-treated BMCMCs from WT or CCRL2 KO mice pre-incubated with or without DNP-specific IgE, and tested in the presence or absence of DNP-HSA. Cell proliferation was measured by tritiated thymidine incorporation. For B, C, and D, n=7 KO, n=4 WT, mean±SD. For Panel E, the mean of triplicate measurements±SD is shown for a representative data set of 3 experiments (each of which gave similar results).

FIG. 4. Mast cell-expressed mCCRL2 is required for maximal tissue swelling and numbers of dermal leukocytes in passive cutaneous anaphylaxis. Panel A: wild type (WT) or CCLR2 knock out (KO) mice were sensitized by injection of 50 ng anti-DNP IgE into left ear skin (with vehicle injection into right ear skin as the control). The mice were challenged by i.v. injection of DNP-HSA (200 μg/mouse, i.v.) the next day, and ear swelling was measured at the indicated time points, mean±SEM, n=3 experiments (a total of 21 KO and 16 WT mice per group), *p<0.005 by ANOVA comparing swelling in WT vs. KO ears sensitized with antigen specific IgE. Panels B-D: the ears of mast cell deficient Kit^{W-sh/Wsh} mice were engrafted with bone marrow-derived cultured mast cells (BMCMCs) from either WT or mCCRL2 KO mice. 6-8 weeks later, the mice were sensitized (5 ng IgE/left ear, with vehicle into the right ear as the control), challenged with specific antigen (200 μg DNP-HSA, i.v.), and assessed for (Panel B) tissue swelling as described in part (A), and for numbers of mast cells (Panel C) or leukocytes (Panel D) per mm² of dermis. Data shown as mean±SEM, n=3 experiments, 15 total mice per group in Panel B and the numbers of mice sampled for histological data shown in Panel C and Panel D. *p<0.001 by ANOVA comparing swelling in mCCRL2 KO BMCMC- vs. WT BMCMC-engrafted ears sensitized with antigen specific IgE. Panel C: enumeration of mast cells present in the dermis of ear skin in engrafted animals from Panel B following elicitation of PCA (IgE) or in vehicle-injected control (vehicle) ears. **p<0.005 by Student's t-test vs. values for the vehicle-injected ears in the corresponding WT BMCMC- or KO BMCMC-engrafted Kit^{W-sh/Wsh} mice. Panel D: numbers of leukocytes per mm² of dermis, assessed in formalin-fixed, paraffin-embedded, hematoxylin and eosin-stained sections of mice from Panels B and C. ***p<0.0001 by the Mann Whitney U-test vs. corresponding values for the vehicle-injected ears in WT BMCMC- or KO BMCMC-engrafted Kit^{W-sh/Wsh} mice. The numbers over the bars for vehicle-injected mice are the mean values.

FIG. 5. Histologic features of IgE-dependent PCA reactions in WT BMCMC- vs. KO BMCMC-engrafted Kit^{W-sh/Wsh} mice. Histological sections of ear skin from WT BMCMC-engrafted Kit^{W-sh/Wsh} mice (Panels A-C) and KO BMCMC-engrafted Kit^{W-sh/Wsh} mice (Panels D-F) from the same group shown in FIG. 4, Panel D show no evidence of inflammation in ears analyzed 6 h after injection of vehicle (Panels A and D), but evidence of tissue swelling and increased numbers of leukocytes, consisting predominantly of polymorphonuclear leukocytes (some indicated by arrowheads in Panels C and F and occasional mononuclear cells (indicated by an arrow in Panel C), at 6 h after antigen challenge in both WT BMCMC-engrafted Kit^{W-sh/Wsh} mice (Panels B and C) and KO BMCMC-engrafted Kit^{W-sh/Wsh} mice (Panels E and F). Hematoxylin and eosin stain; scale bars=50 μm.

FIG. 6. CCRL2 binds chemerin. Panel A: chemerin blocks anti-CCRL2 mAb binding. Various concentrations of human chemerin or CCL2 were incubated with total peritoneal mast cells on ice for 5 minutes, followed by incubation with CCRL2 specific mAb BZ2E3 or anti-IgE and detected with secondary anti-rat PE or anti-mouse IgE PE. (B-C) Radiolabeled chemerin binding. Panel B: displacement of iodinated chemerin (residues 21-148) binding to mCMKLR1, huCCRL2, and mCCRL2 by full-length chemerin. Panel C: saturation binding of ¹²⁵I-chemerin₂₁₋₁₄₈ to mCCRL2-transfected cells. Panel D: immunofluorescence-based chemerin binding. Various concentrations of untagged serum form chemerin were incubated with mCCRL2-HA, huCCRL2-HA, mCRTH2-HA, or mCMKLR1-HA L1.2 transfectants in the presence of 10 nM His₈-tagged serum form chemerin. Samples were incubated on ice for 30 min Secondary anti-His₆ PE was added to detect levels of bound His₈-tagged chemerin, and MFI values are displayed. Mean MFI±range of duplicate staining wells are shown. Panel E: mast cell binding. 1000 nM untagged chemerin isoforms were incubated with total peritoneal cells from either WT or CCRL2 KO mice in the presence or absence of 10 nM His₈-tagged chemerin isoforms. Secondary anti-His₆ PE was added to detect levels of bound His₈-tagged chemerin. SSC^{high} F4/80⁻ c-kit⁺ mast cells were analyzed. A representative data set of the 3 (for Panels B, D, and E) or 2 experiments (for Panels A and C) performed, each of which gave similar results, are shown.

FIG. 7. Chemerin:CCRL2 binding does not trigger intracellular calcium mobilization or chemotaxis. Panel A: mCCRL2 and mCMKLR1 L1.2 transfectants were loaded with Fluo-4, treated with chemerin and/or CXCL12 at the indicated times, and examined for intracellular calcium mobilization. Panel B: mouse peritoneal mast cells were enriched by Nycoprep density centrifugation, loaded with Fura-2 and Fluo-4, and assayed for calcium mobilization. 1000 nM chemerin and 100 nM ATP were added as indicated. Panel C: mCCRL2-HA, huCCRL2-HA, and mCMKLR1-HA L1.2 transfectants were tested for transwell chemotaxis to various concentrations of chemerin. The mean±range of duplicate wells is shown. Panel D: mouse peritoneal mast cells were assayed for in vitro chemotaxis to various concentrations of SCF and chemerin. Mast cells were identified by gating on SSC^{high} CD11b⁻ c-kit⁺ cells. The mean±SD of triplicate wells is shown for an individual experiment. A representative data set of the 3 experiments performed, each of which gave similar results, is shown for all parts of this figure.

FIG. 8. CCRL2 can increase local chemerin concentrations. Panel A: Chemerin does not trigger CCRL2 receptor internalization. mCCRL2-HA, huCCRL2-HA, and mCMKLR1-HA L1.2 transfectants were stained with anti-HA mAb and then incubated with or without 100 nM chemerin for 15 min at the indicated temperatures. Panel B: mCCRL2 is not rapidly constitutively internalized mCCRL2-HA and mCMKLR1-HA L1.2 transfectants were incubated with primary anti-HA mAb, incubated for the indicated times at 37° C., and then stained with secondary anti-mIgG1 PE. mCMKLR1 cells incubated with 100 nM serum form chemerin served as a positive control. Panels C and D: Chemerin is not rapidly internalized mCCRL2-HA L1.2 transfectants (Panel C) or total peritoneal exudate cells (Panel D) were incubated with 10 nM His₈-tagged serum form chemerin and anti-His₆ PE for 1 h on ice, and then shifted to 37° C. At the indicated time points, the cells were then washed with either PBS or acid wash buffer. Mast cells were identified by gating on SSC^{high} F4/80⁻ c-kit⁺ cells in Panel D. Panel E: CCRL2 can sequester chemerin from solution. 2 nM serum form chemerin was incubated with the indicated transfectant lines (or media alone) for 15 minutes at 37° C. The cells were removed by centrifugation, and the conditioned media was tested in transwell chemotaxis using mCMKLR1HA/L1.2 responder cells. The mean±SD of triplicate wells for an individual experiment is shown. Panel F: CCRL2 can increase local concentrations of bioactive chemerin. mCCRL2-HA or empty vector pcDNA3 L1.2 transfectants were pre-loaded with 1000 nM serum form chemerin and washed with PBS. mCMKLR1/L1.2 loaded with Fluo-4 served as responder cells. The intracellular calcium mobilization in the responder cells was measured over time as loaded cells or purified chemoattractant was added. Note that different scales are used on either side of the broken-axis indicator. A representative data set of the 3 experiments performed, each of which gave similar results, is shown for all parts of this figure.

FIG. 9. Proposed model of presentation of chemerin by CCRL2 to CMKLR1. Panel A: chemerin binds to CCRL2 leaving the C-terminal peptide sequence free. The carboxyl-terminal domain of chemerin is critical for transducing intracellular signals and interacts directly with CMKLR1. CCRL2 may thus allow direct presentation of bound chemerin to adjacent CMKLR1-expressing cells. Panel B: Alternatively, CCRL2 may concentrate the ligand for proteolytic processing by activated mast cells or macrophages, enhancing the local production of the active form that could then act as a chemoattractant following release from the cell surface.

FIG. 10. Blood lymphocytes, BM neutrophils, and peritoneal macrophages do not detectably express mCCRL2. A representative data set of the 3 experiments, each of which gave similar results, is shown.

FIG. 11. mCCRL2 is upregulated on macrophages activated by specific cytokines and/or TLR ligands. Panel A: Freshly isolated peritoneal macrophages were cultured for 24 h with various stimuli as indicated. A representative data set of the 3 experiments performed, each of which gave similar results, is shown. Panel B: The promoter regions of CCRL2 contain interferon-stimulated response element (ISRE) sequences that are conserved across species (ISRE: Human: SEQ ID NO:14, Chimpanzee: SEQ ID NO:15, Mouse SEQ ID NO:16, Rat: SEQ ID NO:17, Canine: SEQ ID NO:18) (TATA: Human: SEQ ID NO:19, Chimpanzee: SEQ ID NO:20, Mouse SEQ ID NO:21, Rat: SEQ ID NO:22, Canine: SEQ ID NO:23).

FIG. 12. mCCRL2 KO mice display a normal contact hypersensitivity response to FITC. Mice were sensitized by application of 2% FITC (suspended in acetone-dibutyl phthalate) to the shaved abdomen. Five d later, the mice were challenged by application of 0.5% FITC to the left ear, or vehicle alone to the right ear. Ear swelling was measured at the indicated time points. N=7 KO, n=3 WT, 2 het, mean±SEM.

FIG. 13. mCCRL2 is dispensable for maximal tissue swelling in high dose IgE-mediated passive cutaneous anaphylaxis. Panel A: Mice were sensitized by injection of 150 ng anti-DNP IgE into left ear skin (with vehicle injection into right ear skin as the control). The mice were challenged by i.v. injection of DNP-HSA (200 μg/mouse) the next day, and ear swelling was measured at the indicated time points, mean±SEM, n=2 experiments (12 total KO and WT mice per group); NS, not significant (p>0.05) by ANOVA comparing swelling in WT vs. KO ears sensitized with antigen specific IgE. Panel B: The ears of mast cell deficient Kit^{W-sh/Wsh} mice were engrafted with bone marrow-derived cultured mast cells from either WT or mCCRL2 KO mice. 6-8 weeks later, the mice were sensitized (50 ng IgE), challenged (200 μg DNP-HSA), and monitored as described in Panel A. mean±SEM, 5 total mice per group; NS, not significant (p>0.05) by ANOVA comparing swelling in mCCRL2 KO vs. WT BMCMC reconstituted ears sensitized with antigen specific IgE. Panel C: Numbers of leukocytes per mm² of dermis, assessed in formalin-fixed, paraffin-embedded, hematoxylin and eosin-stained sections of mice from Panel B. *p<0.03 and †† p<0.01 by the Mann Whitney U-test (2-tailed) vs. the corresponding values for the vehicle-injected ears in the corresponding WT BMCMC- or KO BMCMC-engrafted Kit^{W-sh/Wsh} mice, respectively. The numbers over the bars for vehicle-injected mice are the mean values.

FIG. 14. Histologic features of high dose IgE-dependent PCA reactions in WT BMCMC- vs. KO BMCMC-engrafted Kit^{W-sh/Wsh} mice. Histological sections of ear skin from WT BMCMC-engrafted Kit^{W-sh/Wsh} mice (Panels A-C) and KO BMCMC-engrafted Kit^{W-sh/Wsh} mice (Panels D-F) from the same group shown in Figure S4C show no evidence of inflammation in ears analyzed 6 h after injection of vehicle (Panel A and D), but evidence of tissue swelling and increased numbers of leukocytes, consisting predominantly of polymorphonuclear leukocytes (some indicated by arrowheads in Panels C and F and occasional mononuclear cells (indicated by an arrow in Panel F), at 6 h after antigen challenge in both WT BMCMC-engrafted Kit^{W-sh/Wsh} mice (Panels B and C) and KO BMCMC-engrafted Kit^{W-sh/Wsh} mice (Panels E and F). Hematoxylin and eosin stain; scale bars=50 μm.

FIG. 15. mCCRL2/L1.2 transfectants do not migrate to CCL2, CCL5, CCL7, or CCL8 in in vitro transwell chemotaxis. Left panel shows mouse CD11b⁺ peritoneal cells were used as positive controls to demonstrate functional activity of the chemokines tested. Right panel shows mCCRL2/L1.2 cells were tested for chemotactic responses to a range of doses of the indicated chemokines. CCL19/CXCL12 were used as a positive control to demonstrate functional migratory responses by mCCRL2/L1.2 cells (through endogenous expression of CCR7 and CXCR4 by L1.2 cells). A representative experiment (mean±range of duplicate wells) of the 3 performed, each of which gave similar results, is shown.

FIG. 16. Lack of heterologous displacement of chemerin by other chemoattractants. mCCRL2/L1.2 transfectants were co-incubated with 10 nM tagged chemerin and 100-fold excess untagged chemoattractants. Secondary anti-His₆ PE was used to detect levels of bound chemerin. The horizontal bar at MFI=77 indicates the fluorescence intensity of cells incubated with tagged chemerin/secondary anti-His₈ PE in the absence of untagged attractants. The mean±SEM of n=3 experiments is displayed. *p<0.005 comparing the MFI of cells incubated in the presence of tagged chemerin±untagged chemerin.

FIG. 17. Radioligand binding competition. Panel A: Displacement of iodinated chemerin (residues 21-148) binding to mCMKLR1 and mCCRL2 by His₈-tagged chemerin. Panel B: Displacement of iodinated chemerin (residues 21-148) binding to mCMKLR1 and mCCRL2 by bioactive carboxyl-terminal chemerin peptide (YFPGQFAFS). Regression analysis of the binding in part (Panel B) to mCCRL2 failed to fit a curve to the data with R²>0.8 (R²=0.66); thus the EC₅₀ could not be determined (N.D.). A representative experiment (mean±SD of triplicate wells) of the 3 performed, each of which gave similar results, is shown for each part.

FIG. 18. Chemerin and/or CCL2 do not trigger intracellular calcium mobilization in CCRL2/HEK293 transfectants. Panel A and B: mCCRL2 HEK293 transfectants were loaded with Fluo-4, sequentially treated with chemerin, CCL2, or ionomycin at the indicated times, and examined for intracellular calcium mobilization. Panel C: THP1 cells were loaded with Fluo-4, treated with CCL2 at the indicated time, and examined for intracellular calcium mobilization. Panel D: mCMKLR1 HEK293 transfectants were loaded with Fluo-4, treated with chemerin at the indicated time, and examined for intracellular calcium mobilization. A representative data set of the 3 experiments performed, each of which gave similar results, is shown for all parts of this figure.

FIG. 19. CCRL2 amino-terminal sequence alignment. The predicted amino-terminal domains of mouse (SEQ ID NO:24) and human (SEQ ID NO:25) CCRL2 were aligned using Clustal W.

FIG. 20. Freshly isolated peritoneal mast cells do not express CMKLR1. Freshly isolated peritoneal leukocytes were harvested and mCMKLR1 expression was evaluated on SSC^{high} F4/80⁻ c-kit⁺ mast cells. One representative data set of the at least 3 experiments, each of which gave similar results, is shown.

FIG. 21. mRNA expression of mCCRL2. A mouse RNA array was probed with mCCRL2 cDNA.

FIG. 22. CCRL2 is upregulated on TNFα-treated bEND3 endothelioma cells and binds chemerin. bEND3 cells were grown to 95-100% confluence and treated for 24 hours with 20 ng/ml TNFα. Panel A. Robust upregulation of CCRL2 RNA by microarray analysis (Affymetrix®). Panel B. TNFα-treated bEND3 cells acquire CCRL2 surface protein (but not CMKLR1 expression) as determined by mAb staining and flow cytometry. Panel C. Radiolabeled chemerin (residues 21-148) binds to bEND3 cells treated with TNFα. Displacement of iodinated chemerin (residues 21-148) by “cold” serum-form chemerin. CCRL2/L1.2 and empty vector pcDNA3/L1.2 transfectants are shown as controls. The mean+/−SD of triplicate wells is shown for an individual experiment. A representative data set of 2 performed with similar results is shown for parts B and C, and n=1 for part A.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the compound” includes reference to one or more compounds and equivalents thereof known to those skilled in the art, and so forth.

Definitions used herein include, BMCMCs, bone marrow-derived cultured mast cells; CCRL2, chemokine (CC motif) receptor-like 2; CMKLR1, chemokine-like receptor 1; DNP-HSA, 2,4-dinitrophenyl-conjugated human serum albumin; GPCR, G-protein coupled receptor; HA, hemagglutinin; MFI, mean fluorescence intensity; PCA, passive cutaneous anaphylaxis; PEC, peritoneal exudate cells.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The invention provides a method of detecting a candidate agent that modulates the activity of CCRL2. The term “modulate” includes any of the ways mentioned herein in which the agent of the invention is able to modulate CCRL2. This includes upregulation or downregulation of CCRL2 expression, upregulation or downregulation of CCRL2 degradation, stimulation or inhibition of CCRL2 receptor activity, including potentiation of CCRL2 activity in response to a chemerin polypeptide. The ability of a candidate agent to modulate the activity or expression of CCRL2 may be determined by contacting a CCRL2 polypeptide with the agent under conditions that, in the absence of the candidate agent, permit activity or expression of CCRL2, for example in the presence of a chemerin polypeptide, and comparing CCRL2 activity in the presence and absence of the candidate agent. Preferably, the modulation is a correction of aberrant CCRL2 activity or expression. CCRL2 activity is typically activation of a G-protein mediated signaling pathway. The G-protein may be any G-protein that is coupled to the CCRL2 polypeptide.

The methods of detecting an agent that modulates the activity of a CCRL2 polypeptide may be carried out in vitro (inside or outside a cell) or in vivo. In one embodiment the methods are carried out in or on a cell, cell culture or cell extract which comprises a CCRL2 polypeptide or expresses a CCRL2 polynucleotide. The cell may be one in which the CCRL2 polypeptide is naturally expressed, such as an endothelial cell or a mast cell as described herein. Alternatively, the cell may be a cell that is transformed with a CCRL2 polynucleotide and expresses a CCRL2 polypeptide. Suitable cells include transient, or preferably stable higher eukaryotic cell lines, such as mammalian cells or insect cells, lower eukaryotic cells, such as yeast, or prokaryotic cells such as bacterial cells. Particular examples of cell lines include mammalian HEK293T, CHO, HeLa and COS cells. Preferably the cell line selected will be one which is not only stable, but also allows for mature glycosylation of a polypeptide. Expression of a CCRL2 polypeptide may also be achieved in transformed oocytes.

In another embodiment, the methods are carried out in or on a liposome comprising a CCRL2 polypeptide. Methods for the preparation of liposomes are well known in the art (Woodle and Papahadjopoulos, Methods Enzymol., 1989; 171: 193-217).

In a further embodiment, the methods are carried out in or on virus-induced budding membranes comprising a CCRL2 polypeptide. Methods for the preparation of virus-induced budding membranes are well known in the art (for example, Luan et al., Biochemistry, 1995; 34(31): 9874-9883). Viruses may be used to induce budding in cells expressing a CCRL2 polypeptide naturally or cells transformed (transfected) with a CCRL2 polynucleotide.

In a further embodiment, the methods are carried out in or on artificial lipid bilayers. Methods for the preparation of artificial lipid bilayers are well known in the art (Sackmann and Tanaka, Trends Biotechnol., 2000; 18: 58-64; and Karlsson and Lofas, Anal. Biochem., 2002; 300: 132-138). A CCRL2 polypeptide may be integrated into the artificial membrane when the membrane is fabricated.

In a yet further embodiment, the methods are carried out in or on a membrane fraction comprising the CCRL2 polypeptide. A membrane fraction is a preparation of cellular lipid membranes in which some, for example at least 5% or 10%, of the non-membrane-associated elements have been removed. Membrane-associated elements are cellular constituents that are integrated into the lipid membrane or cellular constituents physically associated with a component integrated into the lipid membrane. Methods for the preparation of cellular membrane fractions are well known in the art (for example, Hubbard and Cohn, 1975, J. Cell. Biol., 64; 461-479). A membrane fraction comprising the CCRL2 polypeptide may be prepared from cells expressing a CCRL2 polypeptide naturally or cell transformed (transfected) with a CCRL2 polynucleotide. Alternatively, a CCRL2 polypeptide may be integrated into a membrane preparation by dilution of a detergent solution of the CCRL2 polypeptide (for example, Salamon et al., 1996, Biophys. J., 71: 283-294).

The methods for identifying an agent that modulates the activity of a CCRL2 polypeptide are carried out using a candidate agent. The method typically comprises using one or more candidate agents, for example 1, 2, 3, 4, 5, 10, 15, 20 or 30 or more candidate agents. A candidate agent is a candidate compound being evaluated for the ability to modulate the activity of CCRL2 by the methods of the invention. Candidate agents can be natural or synthetic compounds, including, for example, small molecules, compounds contained in extracts of animal, plant, bacterial or fungal cells, as well as conditioned medium from such cells. Suitable candidate agents which may be tested in the above screening methods include antibody agents (for example, monoclonal and polyclonal antibodies, single chain antibodies, chimeric antibodies and CDR-grafted antibodies) or aptamer agents. The antibody agent may have binding affinity for the CCRL2 receptor or for a chemerin polypeptide. Furthermore, combinatorial libraries, defined chemical identities, peptide and peptide mimetics, oligonucleotides and natural agent libraries, such as display libraries (e.g. phage display libraries) may also be tested. Oligonucleotide libraries, such as aptamer libraries may be tested.

The candidate agents may be chemical compounds, which are typically derived from synthesis around small molecules.

The candidate agent may be derived from or contained in an environmental sample, a natural extract of animal, insect, marine organism, plant, yeast or bacterial cells or tissues, a clinical sample, a synthetic sample, or a conditioned medium from recombinant cells or a fermentation process. The candidate agent may also be derived from or contained in a tissue sample which comprises a body fluid and/or cells of an individual and may, for example, be obtained using a swab, such as a mouth swab. The candidate agent may be derived from or contained in a blood, urine, saliva, skin, cheek cell or hair root sample.

Batches of the candidate agents may be used in an initial screen of, for example, ten candidate agents per reaction, and the candidate agents of batches which show modulation tested individually. Where a batch of agents shows CCRL2 modulatory activity the test agents may be tested in smaller batched or individually to identify the agent having modulatory activity.

Exemplary candidate agents are polypeptides, antibodies or antigen-binding fragments thereof, lipids, carbohydrates, nucleic acids and chemical compounds.

The methods of the invention detect agents that modulate the activity of a CCRL2 polypeptide by determining or assaying the effect of a candidate agent on an activity of the CCRL2 polypeptide such as ligand binding, signalling activity or chemotactic activity. The methods of the invention are carried out under conditions which, in the absence of the candidate agent, permit the binding of a chemerin polypeptide to a CCRL2 polypeptide. These conditions are, for example, the temperature, salt concentration, pH and protein concentration under which a chemerin polypeptide binds to a CCRL2 polypeptide. Exact binding conditions will vary depending upon the nature of the assay, for example, whether the assay uses viable cells or only membrane fraction of cells. However, because CCRL2 is a cell surface receptor and chemerin is secreted polypeptides that interact with the extracellular domain of CCRL2, preferred conditions will generally include physiological salt concentration (approximately 90 mM) and pH (about 7.0 to 8.0). Temperatures for binding may vary from 4° C. to 37° C., but is preferably 4° C. The concentration of reactants in the binding assay will also vary, but will preferably be from about 0.1 pM to about 10 μM.

In one embodiment of the invention, the effect of the test sample on the binding of the CCRL2 polypeptide to chemerin is monitored. Any suitable binding assay format can be used to monitor binding and detect any effect. The effect may be measured as a decrease in the binding between chemerin and a CCRL2 polypeptide. A decrease of at least 10%, at least 20%, at least 30%, at least 40%, at least 50%, at least 60%, at least 70% or at least 80% in the binding between chemerin and a CCRL2 polypeptide measured in any given assay indicates that the candidate agent modulates the activity of CCRL2.

Exemplary assays for monitoring any candidate agent-induced changes in the binding between chemerin and a CCRL2 polypeptide include label displacement, surface plasmon resonance, fluorescence resonance energy transfer, fluorescence quenching, fluorescence polarization and radioligand binding assays.

Label displacement involves contacting a CCRL2 polypeptide with a detectably labeled chemerin in the presence or absence of increasing concentrations of a candidate agent. To calibrate the assay, control competition reactions using increasing concentrations of an unlabelled chemerin may be carried out. After contact, bound, labeled chemerin is measured using a method appropriate for the given label (for example scintillation counting, enzyme assay or fluorescence). Preferred labels include radioisotopes such as tritium or iodine or any other suitable radionucleotide. Candidate agents are considered to bind specifically to a CCRL2 polypeptide if they displace 50% of labeled chemerin at a concentration of 10 μM or less (EC₅₀ is 10 μM or less).

Surface plasmon resonance measures binding between the two molecules by the change in mass near an immobilized sensor caused by the binding or loss of binding of chemerin to a CCRL2 polypeptide immobilized in a membrane on the sensor. The change in mass is measured as resonance units versus time after injection or removal of the ligand or candidate agent and is measured using a Biacore Biosensor (Biacore AB). A CCRL2 polypeptide may be immobilized on a sensor chip in a thin film lipid membrane according to methods described (Salamon et al., 1996, Biophys J. 71: 283-294). Generally, a candidate agent may be administered to chemerin pre-bound to an immobilized CCRL2 polypeptide and displacement of the ligand measured. Alternatively, chemerin may be administered to a candidate agent pre-bound to an immobilized CCRL2 polypeptide.

Fluorescence resonance energy transfer (FRET) is a quantum mechanical phenomenon that occurs between a fluorescence donor (D) and a fluorescence acceptor (A) in close proximity to each other if the emission spectrum of D overlaps with the excitation spectrum of A. Generally, the chemerin and the CCRL2 polypeptide are labeled with a complementary pair of donor and acceptor fluorophores. The fluorescence emitted upon excitation of the donor fluorophore will have a different wavelength when chemerin and CCRL2 polypeptide are bound than when they are not bound. Quantitation of bound versus unbound polypeptides can be carried out by measurement of emission intensity at each wavelength. Donor:Acceptor pairs of fluorophores with which to label the polypeptides are well known in the art. Preferred fluorophores are Cyan Fluorescent Protein (CFP, Donor) and Yellow Fluorescent Protein (YFP, Acceptor).

Fluorescence quenching involves labeling one molecule of the binding pair (chemerin and CCRL2 polypeptide) with a fluorophore while labeling the other with a molecule that quenches the fluorescence of the fluorophore when the pair bind. A change in fluorescence upon excitation may be used to measure a change in the binding between chemerin and CCRL2. An increase in fluorescence suggests that the binding between chemerin and CCRL2 polypeptide is decreased.

Fluorescence polarization measures the polarization of a fluorescently-labeled chemerin. The fluorescence polarization value for a fluorescently-labeled chemerin will change, and generally increase, when the ligand binds to a CCRL2 polypeptide. A decrease in the polarization value is typically indicative of a decrease in binding between chemerin and CCRL2 polypeptide. Fluorescence polarization is preferable when the candidate agent is a small molecule.

Large scale, high throughput screening of small candidate agents or libraries of such agents may be screened using biosensor assays. ICS biosensors have been described by AMBRI (Australian Membrane Biotechnology Research Institute; available on the worldwide web at www.ambri.com.au/). The binding of a ligand for CCRL2 to CCRL2 is coupled to the closing of gramacidin-facilitated ion channels in a membrane bilayer of the biosensors. As a result, the biosensor may measure binding between chemerin and CCRL2 polypeptide and therefore any changes in binding upon introduction of a candidate agent.

Agents that interfere with or displace binding of chemerin from a CCRL2 polypeptide may be agonists, partial agonists, antagonists or inverse agonists of CCRL2 activity. Functional analysis can be performed on agents identified according to the invention to determine whether they are an agonist, partial agonist, antagonist or inverse agonist. For agonist screening, a CCRL2 polypeptide is contacted with agent and the signaling activity of CCRL2 measured as described below. In certain embodiment, the signaling activity will be in the context of CCRL2 presentation of chemerin to CMKLR1+ cells. An agonist or partial agonist will have a maximal activity corresponding to at least 10% of the maximal activity of chemerin. The agonist or partial agonist will preferably have 50%, 75%, 100% activity of chemerin or 2-fold, 5-fold, 10-fold or more activity than chemerin. For antagonist or inverse agonist screening, CCRL2 polypeptides are assayed for signaling activity in the presence of chemerin, with or without a candidate compound. Antagonists or inverse agonists will reduce the level of ligand-stimulated receptor activity by at least 10%, compared to reactions lacking the antagonist or inverse agonist. For inverse agonist screening, constitutive CCRL2 activity is assayed in the presence and absence of a candidate compound. Inverse agonists are compounds that reduce the constitutive activity of the receptor by at least 10%. Constitutive activity of a CCRL2 polypeptide may be achieved by overexpression by placing, for example, placing it under the control of a strong constitutive promoter such as the CMV early promoter. Alternatively, constitutive activity may be achieved by certain mutations of conserved G-protein coupled receptor amino acids or amino acid domains (for example, Kjelsberg et al., 1992, J. Biol. Chem. 267:1430-1430; Ren et al., 1993, J. Biol. Chem. 268:16483-16487; and Samama et al., 1993, J. Biol. Chem. 268:4625-4636).

In another embodiment of the invention, the effect of a test sample on the signaling activity of a CCRL2 polypeptide is monitored. The signaling activity of CCRL2 is induced by chemerin. Any suitable signaling assay format may be used for monitoring signaling activity and detecting any effect. The effect may be measured as a change in chemerin-induced signaling activity of CCRL2. A change refers to an increase or a decrease in the signaling activity. A change of at least 10%, at least 20%, at least 30%, at least 40%, at least 50%, at least 60%, at least 70% or at least 80% in the signaling activity a CCRL2 polypeptide measured in any given assay indicates that the candidate agent modulates the activity of CCRL2.

The signalling activity of a CCLR2 polypeptide may be monitored by measuring the level of activation of a G protein by the CCLR2 polypeptide. The level of activation of a G protein by CCRL2 may be monitored by measuring the turnover of guanosine derivatives, the activity of guanosine triphosphatase (GTPase) or level of downstream second messenger molecules. Guanosine derivatives are involved in the cyclic reaction of activation and inactivation of G proteins include guanosine diphosphate (GDP) and guanosine triphosphate (GTP). Second messenger molecules are generated or caused to alter in concentration by the activation of a G protein. Examples include but are not limited to cyclic adenine monophosphate (cAMP), cyclic guanosine monophosphate (cGMP), diacylglycerol (DAG), inositol triphosphate (IP₃) and intracellular calcium.

Exemplary methods of monitoring signaling activity include measuring guanosine nucleotide binding, GTPase activity, adenylate cyclase activity, cAMP, Protein Kinase C activity, phosphatidylinositol breakdown, diacylglycerol, inositol triphosphate, intracellular calcium, MAP kinase activity and reporter gene expression. In all assays, potential non-specific effects of the candidate agent may be excluded by carrying out control assays using cells or membranes that do not comprise a CCRL2 polypeptide.

GTP binds to membrane-associated G proteins upon activation by a receptor such as a CCRL2 polypeptide. CCRL2 signaling activity may therefore be assayed by measuring the binding of GTP to cell membranes containing receptors (Traynor and Nahorski, 1995, Mol. Pharmacol. 47: 848-854). Generally, GTP is labeled with a suitable detectable moiety and measured by an appropriate detection system.

G proteins comprise a GTPase which hydrolyses GTP to form GDP and inactivates the G protein. GTPase activity is therefore a measure of G protein and therefore CCRL2 activity. GTPase activity may be measured by methods common in the art. Generally, the method involves incubating the membranes containing a CCRL2 polypeptide with γP-GTP. Active GTPase will release the label as inorganic phosphate which may be detected by scintillation counting.

Another exemplary method of monitoring signaling activity is measuring adenylate cyclase activity (Solomon et al., 1974, Anal. Biochem. 58: 541-548; and Kenimer & Nirenberg, 1981, Mol. Pharmacol. 20: 585-591). The assay may involve the use of labeled cAMP to estimate the activity of the adenylate cyclase enzyme in protein homogenates from cells or membrane comprising a CCRL2 polypeptide.

In some embodiments the method of monitoring signalling activity is the measurement of intracellular cAMP. This may be done using a cAMP radioimmunoassay (RIA) or cAMP binding proteins according to methods known in the art (Horton & Baxendale, 1995, Methods Mol. Biol. 41: 91-105). Intracellular cAMP may be measured using a number of commercially available kits including the High Efficiency Fluorescence Polarization-based homogeneous assay (LJL Biosystems and NEN Life Science Products).

In other embodiments, the methods of monitoring signaling activity measure receptor induced breakdown of phospholipids (especially phosphatidylinositol) to generate the second messengers DAG and/or IP₃. Methods of measuring each of these are well known in the art (for example, Phospholipid Signaling Protocols, edited by Ian M. Bird. Totowa, N.J., Humana Press, 1998; and Rudolph et al., 1999, J. Biol. Chem. 274: 11824-11831).

In yet another embodiment, the method of monitoring signalling activity measures receptor induced Protein Kinase C (PKC) activity. DAG activates PKC which phosphorylates many target proteins and ultimately results in the transcription of an array of proto-oncogene transcription factor-encoding genes, including c-fos, c-myc and c-jun, proteases; protease inhibitors, including collagenase type I and plasminogen activator inhibitor; and adhesion molecules, including intracellular adhesion molecule I (ICAM I). The activity of PKC may be measured directly by measuring phosphorylation of a substrate peptide, Ac-FKKSFKL-NH2, which derived from the myristoylated alanine-rich protein kinase C substrate protein (MARCKS) (Kikkawa et al., 1982, J. Biol. Chem. 257: 13341-13348). Assays designed to detect increases in gene products induced by PKC can be used to monitor PKC activation and thereby receptor activity. In addition, the activity of a receptor that activates PKC can be monitored through the use of reporter gene constructs driven by the control sequences of genes activated by PKC activation.

In yet another embodiment, the method for monitoring signaling activity measures MAP kinase activity. Several kits are commercially available, including the p38 MAP Kinase assay kit (New England Biolabs (Cat #9820)) and the FlashPlate™ MAP Kinase assay (Perkin-Elmer Life Sciences).

Other exemplary methods of monitoring signaling activity measure changes in the transcription or translation of one or more genes. Generally, assays measure the expression of a reporter gene driven by control sequences, such as promoters and transcription-factor binding sites, responsive to receptor activation. Cells that comprise a CCRL2 polypeptide may be stably transfected with a reporter gene construct containing appropriate control sequences. Assays tend to involve measuring the response of “immediate early” genes which may be rapidly induced, possibly within minutes, of receptor activation. Suitable reporter genes include, but are not limited to, luciferase, CAT, GFP, β-lactamase or β-galactosidase. An example of a control sequence that may be used in a reporter gene assay are those of the c-fos gene. The induction of c-fos expression is extremely rapid, often within minutes, of receptor activation. The c-fos regulatory elements are well known in the art (Verma et al, 1987, Cell 51: 513-514). A further example of a control sequence that may be used in a reporter gene assay are those recognized by CREB (cyclic AMP responsive element binding protein). Other examples of control sequences that may be used in a reporter gene assay include, but are not limited to, the vasoactive intestinal peptide (VIP) gene promoter (Fink et al., 1988, Proc. Natl. Acad. Sci. 85:6662-6666); the somatostatin gene promoter (Montminy et al., 1986, Proc. Natl. Acad. Sci. 83:6682-6686); the proenkephalin promoter (Comb et al., 1986, Nature 323:353-356); the phosphoenolpyruvate carboxy-kinase (PEPCK) gene promoter (Short et al., 1986, J. Biol. Chem. 261:9721-9726); and transcriptional control elements responsive to the AP-1 transcription factor (Lee et al, 1987, Nature 325: 368-372; and Lee et al., 1987, Cell 49: 741-752) or NF-κB activity (Hiscott et al., 1993, Mol. Cell. Biol. 13: 6231-6240). Although for other signaling activity assays, a change of at least 10% in the presence of a candidate agent indicates that it modulates CCRL2, the transcriptional reporter assay requires at least a two-fold increase in signal to indicate the presence of a positive agent. As with other assays, a negative agent is indicated by at a 10% decrease in signal in the reporter gene expression assay.

The ability of a candidate agent identified by a method of the invention to modulate the signaling activity of a CCRL2 polypeptide may be further confirmed or analyzed. This functional analysis is described in detail above. This analysis typically involves monitoring of the effect of candidate agent alone on the signaling activity of a CCRL2 polypeptide and comparison with the effect of chemerin on the signaling activity of the CCRL2 polypeptide. Any suitable signaling assay format may be used for determining signaling activity and detecting the effect. The effect may be measured as a change in the signaling activity of CCRL2. The agent may be agonist, partial agonist, antagonist or inverse agonist of CCRL2 activity.

A method of modulating the activity of a CCRL2 polypeptide in a cell is provided by the invention, which method comprises delivering an agent detected according to the invention the cell, such that the activity of CCRL2 is modulated. The cell may be in vivo or in vitro. The delivery of the agent is discussed in more detail below.

A method of treating an inflammatory disease or disorder, a method of treating a disease or disorder associated with enhanced macrophage or mast cell activity and a method of treating an infection are also provided by the invention, which methods comprise administering a therapeutically effective amount of an agent according to the invention to an individual in need thereof.

A method of treating an inflammatory disease or disorder of the invention typically comprises: (i) identifying an agent for the prevention or treatment of an inflammatory disease or disorder by a method according to the invention; and (ii) administering a therapeutically effective amount of an agent detected in (i) to an individual having an inflammatory disease or disorder.

In all the above embodiments, the inflammatory disease or disorder includes, for example, chronic obstructive pulmonary disease (COPD), bronchitis, emphysema, an inflammatory bone disorder, psoriasis, inflammatory bowel disease, an inflammatory brain disorder, atherosclerosis, endometriosis, autoimmune deficiency syndrome (AIDS), lupus erythematosus, allograft rejection, rheumatoid arthritis or allergic inflammation. The inflammatory brain disorder may be multiple sclerosis, or stroke or heamorrhage. The inflammatory bowel disease may be ulcerative colitis or Crohn's disease. The inflammatory bone disorder may be arthritis, including rheumatoid, autoimmune and infectious arthritis. The allergic inflammation may be, for example, asthma or contact dermatitis. The inflammatory disease or disorder may be a CCRL2-related disease or disorder. An inflammatory disease or disorder may be present, or be suspected of being present, in the individual to be treated. The individual is discussed in more detail below.

When administration is for the purpose of treatment, administration may be either for prophylactic or therapeutic purpose. When provided prophylactically, the agent or polypeptide, polynucleotide or antibody is provided in advance of any symptom. The individual may have been identified as having a genetic predisposition to an inflammatory disease or disorder. For example, where the inflammatory disease or disorder is a CCRL2-related disease or disorder, such as inflammatory bowel disease, atherosclerosis, endometriosis or an inflammatory brain disease the individual may have a polymorphism in the CCRL2 gene which polymorphism is associated with the disease or disorder. The prophylactic administration of the agent or polypeptide, polynucleotide or antibody serves to prevent or attenuate any subsequent symptom. When provided therapeutically the agent or polypeptide, polynucleotide or antibody is provided at or following, preferably shortly after, the onset of a symptom. The therapeutic administration of the agent or polypeptide, polynucleotide or antibody serves to attenuate any actual symptom. Administration and therefore the methods of the invention may be carried out in vivo or in vitro.

The formulation of any of the therapeutic agents mentioned herein, including polypeptides, polynucleotides and antibodies, will depend upon factors such as the nature of the agent and the condition to be treated. Any such agent may be administered or delivered in a variety of dosage forms. It may be administered or delivered orally (e.g. as tablets, troches, lozenges, aqueous or oily suspensions, dispersible powders or granules), parenterally, subcutaneously, intravenously, intramuscularly, intrasternally, transdermally or by infusion or inhalation techniques. The agent may also be administered or delivered as suppositories. A physician will be able to determine the required route of administration or delivery for each particular patient.

Typically the agent is formulated for use with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuff; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

A therapeutically effective amount of agent is administered. A therapeutically effective amount of an agent is an amount that alleviates the symptoms or which prevents or delays the onset of symptoms of an inflammatory disease or disorder.

The dose may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg, preferably from about 0.1 mg/kg to 10 mg/kg of body weight, according to the activity of the specific inhibitor, the age, weight and conditions of the subject to be treated, the type and severity of the disease and the frequency and route of administration. Preferably, daily dosage levels are from 5 mg to 2 g.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Methods and Materials

The following methods and materials were used in the examples below.

Antibodies and Reagents

Anti-mouse -CD11b, -CD11c, -CD14, -CD19, -B220, -F4/80, -Gr1, -TCRβ-c-kit, -CD49b, -TER119 dye-linked mAb were obtained from eBioscience (San Diego, Calif., USA), BD PharMingen (San Diego, Calif., USA), and Serotec (Raleigh, N.C., USA). Anti-rat phycoerythrin (human and mouse adsorbed) was purchased from BD Pharmingen, anti-His₆ phycoerythrin was purchased from R&D Systems (Minneapolis, Minn., USA), purified Fc block (mouse anti-mouse CD16.2/32.2) was purchased from Caltag (Burlingame, Calif., USA), anti-mCMKLR1 (BZ194) was prepared in house, and mouse IgG, rat IgG, and goat serum were purchased from Sigma (St. Louis, Mo., USA). CCL2-5,7-9,11,16,17,19-22,25,28; CXCL1,2,3,9,10,12,13,16; IL-4; GM-CSF; and Flt-3 ligand were purchased from R&D Systems. Mouse CCL2 biotinylated fluorokine kit was purchased from R&D Systems. CMFDA, Fluo-4-acetoxymethyl (AM), and Pluronic acid F-127 (reconstituted in DMSO) were purchased from Molecular Probes (Eugene, Oreg., USA). Bioactive chemerin peptide (YFPGQFAFS (SEQ ID NO:01)) was synthesized by the Stanford PAN facility. Phosphothioated CpG oligonucleotides (Bauer, M., V. Redecke, J. W. Ellwart, B. Scherer, J. P. Kremer, H. Wagner, and G. B. Lipford. 2001. Bacterial CpG-DNA triggers activation and maturation of human CD11c−, CD123+ dendritic cells. J Immunol 166:5000-5007) were purchased from Qiagen (Valencia, Calif., USA). polyI:C and fMLP were purchased from Sigma. LPS (E. coli 011:B4-derived) was purchased from List Biologicals (Campbell, Calif., USA). TNFα and IFNγ were purchased from Roche (Penzberg, Germany). Complete and incomplete Freund's adjuvant (CFA and IFA) were purchased from Sigma. Cytokine levels in culture supernatants were measured by using mouse TNFα and IL-6 BD OptEIA™ ELISA Sets (BD PharMingen).

Animals

The Veterans Affairs Palo Alto Health Care System Institutional Animal Care and Use Committee, Palo Alto, Calif., and the Stanford University Administrative Panel on Laboratory Animal Care, Stanford, Calif., approved all animal experiments. CCRL2 KO mice were obtained from Lexicon (The Woodlands, Tex., USA) and backcrossed 4 generations on the C57BL/6 background. Mast cell deficient Kit^{W-sh/Wsh} mice on the C57BL/6 background (Tono, T., T. Tsujimura, U. Koshimizu, T. Kasugai, S. Adachi, K. Isozaki, S, Nishikawa, M. Morimoto, Y. Nishimune, S, Nomura, and et al. 1992. c-kit Gene was not transcribed in cultured mast cells of mast cell-deficient Wsh/Wsh mice that have a normal number of erythrocytes and a normal c-kit coding region. Blood 80:1448-1453) were kindly provided by Peter Besmer (Memorial Sloan-Kettering Cancer Center and Cornell University Graduate School of Medical Sciences, NY) and WT C57BL/6 mice were obtained from Taconic (Oxnard, Calif., USA). Wistar Furth rats were obtained from Charles River Laboratories (Wilmington, Mass., USA).

Mammalian Expression Vector Construction and Generation of Stable Cell Lines

The coding regions of mCCRL2, huCCRL2, mCRTH2, and huCCR10 were amplified from genomic DNA with or without an engineered N-terminal hemagglutinin (HA) tag, and cloned into pcDNA3 (Invitrogen, Carlsbad, Calif., USA). Transfectants were generated and stable lines selected in the mouse pre-B lymphoma cell line L1.2 or HEK293 cells as described (Ponath, P. D., S. Qin, T. W. Post, J. Wang, L. Wu, N. P. Gerard, W. Newman, C. Gerard, and C. R. Mackay. 1996. Molecular cloning and characterization of a human eotaxin receptor expressed selectively on eosinophils. J Exp Med 183:2437-2448). mCMKLR1 and empty vector L1.2 transfectants were generated as previously described (Zabel, B. A., A. M. Silverio, and E. C. Butcher. 2005. Chemokine-like receptor 1 expression and chemerin-directed chemotaxis distinguish plasmacytoid from myeloid dendritic cells in human blood. J Immunol 174:244-251). Transfected cells were in some cases treated with 5 mM n-butyric acid (Sigma) for 24 h before experimentation (Palermo, D. P., M. E. DeGraaf, K. R. Marotti, E. Rehberg, and L. E. Post. 1991. Production of analytical quantities of recombinant proteins in Chinese hamster ovary cells using sodium butyrate to elevate gene expression. J Biotechnol 19:35-47).

Generating the anti-mCCRL2 mAbs BZ5B8 and BZ2E3

The immunizing amino-terminal mCCRL2 peptide with the sequence NH₂-MDNYTVAPDDEYDVLILDDYLDNSC-COOH (SEQ ID NO:02)(corresponding to residues 1-24 of mCCRL2, with a non-native carboxyl-terminal cysteine to facilitate conjugation to keyhole limpet hemocyanin, (KLH)) was synthesized by the Stanford Protein and Nucleic Acid Biotechnology Facility and conjugated to KLH according to the manufacturer's specifications (Pierce Biotechnology, Rockford, Ill., USA). Wistar Furth rats were immunized with the mCCRL2 peptide/KLH conjugate first emulsified in CFA, and then subsequently in IFA. Hybridomas producing anti-mCCRL2 mAbs were subcloned, and specificity was confirmed by reactivity with mCCRL2 but not other L1.2 receptor transfectants. An ELISA-based assay (BD Pharmingen) was used to assess the IgG₂ₐκ isotypes of the resulting rat anti-mouse CCRL2 mAbs, designated BZ5B8 and BZ2E3.

Harvesting Mouse Leukocytes

Mice were given a fatal overdose of anesthesia (ketamine/xylazine) as well as an i.p. injection of heparin (100 units, Sigma), and blood was collected by cardiac puncture. Up to 1 mL of blood was added to 5 mL of 2 mM EDTA in PBS, and 6 mL of 2% dextran T500 (Amersham Biosciences, Piscataway, N.J., USA) was added to crosslink red blood cells. The mixture was incubated for 1 hour at 37° C., the supernatant was removed and pelleted, and the cells were resuspended in 5 mL red blood cell lysis buffer (Sigma) and incubated at RT for 5 minutes. The cells were pelleted, and resuspended for use in cell staining. Bone marrow cells were harvested by flushing femurs and tibias with media followed by red blood cell lysis. Peritoneal lavage cells were obtained by i.p. injection of 10 mL PBS, gentle massage of the peritoneal cavity, and collection of the exudate. For some experiments, 500 μl of peritoneal cells (2×10⁶ cells/mL) were incubated for 24 hours with either LPS (1 μg/mL), TNFα (10 ng/mL), IFNγ (100 U/mL), polyI:C (20 μg/mL), CpG (10-100 μg/mL), or TGFβ (5 ng/mL). For mast cell RNA isolation, peritoneal mast cells were enriched for by density centrifugation. Peritoneal exudate cells (˜140 million) were harvested from 9 male WT mice>1 y.o. The cells were resuspended in 10 ml of PBS and underlayed with 5 ml NycoPrep 1.077A (Axis-shield PoC AS, Oslo, Norway). Following centrifugation, ˜140,000 high-density mast cells were recovered at the bottom of the tube (along with ˜100,000 contaminating red blood cells). For functional assays using primary mast cells, peritoneal lavage was performed using Tyrodes solution, and the cells were kept at room temperature throughout harvest.

Cell Sorting and Wright-Giemsa Stain

Mouse peritoneal cells were stained as described and sorted by standard flow cytometric techniques (FACsvantage, BD Biosciences, Mountain View, Calif., USA; flow cytometry was performed at the Stanford University Digestive Disease Center Core Facility, VA Hospital, Palo Alto, Calif., USA). 1-5×10⁴ sorted cells were loaded into cytospin chambers and centrifuged onto glass slides. The slides were stained with Wright-Giemsa dye by standard automated techniques at the VA Hospital Hematology Lab (Palo Alto, Calif., USA) and examined by light microscopy with a 40× objective.

RNA Expression Analysis

RNA from the indicated tissues or cells was extracted using a Qiagen RNeasy kit per the supplier's instructions. Gene expression was determined by quantitative PCR (QPCR) using an Applied Biosystems 7900HT real-time PCR instrument equipped with a 384-well reaction block. 0.3-1.0 μg total RNA was used as template for cDNA synthesis using MMLV Reverse Transcriptase (Applied Biosystems) with oligo dT primers according to the supplier's instructions. The cDNA was diluted and amplified by quantitative PCR in triplicate wells using 10 pmols of gene specific primers in a total volume of 10 μL with Power SYBR Green QPCR Master Mix (Applied Biosystems), according to manufacturer's instructions. CCRL2 gene expression was normalized to cyclophilin A (cycA) levels in each tissue, and displayed relative to CCRL2 expression levels detected in the spleen using the 2^{-ΔΔCT} method (Livak, K. J., and T. D. Schmittgen. 2001. Analysis of relative gene expression data using real-time quantitative PCR and the 2(-Delta Delta C(T)) Method. Methods 25:402-408).

| | |
|---|---|
| mCCRL2 5′ primer: | |
| TTCCAACATCCTCCTCCTTG; | (SEQ ID NO: 03) |
| mCCRL2 3′ primer: | |
| GATGCACGCAACAATACCAC; | (SEQ ID NO: 04) |
| cycA 5′ primer: | |
| GAGCTGTTTGCAGACCAAAGTTC; | (SEQ ID NO: 05) |
| cycA 3′ primer: | |
| CCCTGGCACATGAATCCTGG. | (SEQ ID NO: 06) |

Preparation of Bone Marrow-Derived Cultured Mast Cells (BMCMCS)

Mouse femoral BM cells were cultured in 20% WEHI-3 cell conditioned medium (containing IL-3) for 6-12 weeks, at which time the cells were >98% c-kit^{high} FcεRIα^{high} by flow cytometry analysis.

β-hexosaminidase Release Assay

BMCMCs were sensitized with 10 μg/ml of anti-DNP IgE mAb (H1-ε-26) (Liu, F. T., J. W. Bohn, E. L. Ferry, H. Yamamoto, C. A. Molinaro, L. A. Sherman, N. R. Klinman, and D. H. Katz. 1980. Monoclonal dinitrophenyl-specific murine IgE antibody: preparation, isolation, and characterization. J Immunol 124:2728-2737) by overnight incubation at 37° C. The cells were then washed with Tyrodes buffer (10 mM HEPES pH 7.4, 130 mM NaCl, 5 mM KCl, 1.4 mM CaCl2, 1 mM MgCl2, 0.1% glucose and 0.1% bovine serum albumin [fraction V, SIGMA]), and resuspended at 8×10⁶ cells/ml. 25 μl of a 2× concentration of stimuli (final 0, 1, 10 and 100 ng/ml 2,4-dinitrophenyl-conjugated human serum albumin [DNP-HSA; SIGMA] or 0.1 μg/mg PMA [SIGMA]+1 μg/ml A23187 calcium ionophore [SIGMA]) were added to the wells of 96 well V-bottom plate (Costar), and then 25 μl of 8×10⁶ cells/ml IgE-sensitized BMCMCs were added and incubated at 37° C. for 1 hour. After centrifugation, supernatants were collected. The supernatants from non-stimulated IgE-sensitized BMCMCs treated with 50 μl of 0.5% (v/v) Triton X-100 (SIGMA) were used to determine the maximal (100%) cellular β-hexoaminidase content, to which the experimental samples were normalized. β-hexosaminidase release was determined by enzyme immunoassay with p-nitrophenyl-N-acetyl-β-D-glucosamine (SIGMA) substrate as follows: 10 μl of culture supernatant were added to the wells of a 96 well flat-bottom plate. 50 μl of 1.3 mg/ml p-nitrophenyl-N-acetyl-β-D-glucosamine solution in 100 mM sodium citrate (pH 4.5) was added, and the plate was incubated at room temperature for 15-30 minutes. Next, 140 μl of 200 mM glycine (pH 7.0) was added to stop the reaction and the OD405 was determined.

T-cell:mast Cell Co-Culture

For CD3⁺ T cell purification, a single cell suspension of spleen cells was prepared, and red blood cells were lysed (RBC lysing buffer, Sigma). Spleen cells were incubated with biotinylated anti-mouse B220, Gr-1, CD11b, CD11c, CD49b, Ter119, and c-kit for 20 minutes at 4° C. The cells were then washed and incubated with streptavidin-beads (Miltenyi Biotec) for 20 minutes at 4° C., and washed again and passed through a magnetic cell-sorting column (MACS column; Miltenyi Biotec), yielding>95% CD3⁺ T cells. T cells were co-cultured with mast cells as described previously (Nakae, S., H. Suto, M. Kakurai, J. D. Sedgwick, M. Tsai, and S. J. Galli. 2005. Mast cells enhance T cell activation: Importance of mast cell-derived TNF. Proc Natl Acad Sci USA 102:6467-6472). BMCMCs were sensitized with 1 μg/ml of anti-DNP IgE mAb at 37° C. overnight. After IgE sensitization, BMCMCs were treated with mitomycin C (Sigma; 50 μg per 10⁷ cells) for 15 minutes at 37° C. BMCMCs and T cells were suspended in RPMI 1640 media (Cellgro) supplemented with 50 μM 2-mercaptoethanol (Sigma), 50 μg/ml streptomycin (Invitrogen), 50 U/ml penicillin (Invitrogen) and 10% heat inactivated FCS (Sigma). T cells (0.25×10⁵ cells/well) were plated in a 96 well flat-bottom plate (BD Falcon) coated with 1 μg/ml anti-mouse CD3 (145-2C11; BD PharMingen) or hamster IgG (eBioscience) (in some experiments, “anti-CD3 (−)” indicates the substitution of control IgG for anti-CD3), and mitomycin C-treated, IgE-sensitized or non-sensitized BMCMCs (0.25×10⁵ cells/well) in the presence or absence of 5 ng/ml DNP-HSA at 37° C. for 72 hours. Proliferation was assessed by pulsing with 0.25 μCi [³H]-thymidine (Amersham Bioscience) for 6 hours, harvesting the cells using Harvester 96® Mach IIIM (TOMTEC) and measuring incorporated [³H]-thymidine using a Micro Beta System (Amersham Bioscience).

Passive Cutaneous Anaphylaxis (PCA) Reaction

Experimental PCA was performed as previously described (Wershil, B. K., Z. S. Wang, J. R. Gordon, and S. J. Galli. 1991. Recruitment of neutrophils during IgE-dependent cutaneous late phase reactions in the mouse is mast cell-dependent. Partial inhibition of the reaction with antiserum against tumor necrosis factor-alpha. J Clin Invest 87:446-453), with minor modifications. Mice were injected intradermally with 20 μl of either anti-DNP IgE mAb (H1-ε-26; 5, 50 or 150 ng) in the left ear skin, or vehicle alone (PIPES-HMEM buffer) in the right ear skin. The next day, mice received 200 μl of 1 mg/ml DNP-HSA (200 μg per mouse) intravenously. Ear thickness was measured before and at multiple intervals after DNP-HSA injection with an engineer's microcaliper (Ozaki MFG. CO., LTD., Itabashi, Tokyo, Japan). For BMCMC engraftment experiments, BMCMCs were generated from either WT or LCCR KO mice, and 1×10⁶ cells in 40 μl DMEM were injected into the ear skin of mast cell-deficient Kit^{W-sh/Wsh} mice. 6-8 weeks later the mice were subjected to experimental PCA. After the assay, the mast cells in the ear skin were enumerated in formalin-fixed, paraffin-embedded, toluidine blue-stained sections to evaluate the extent of engraftment; in some experiments, formalin-fixed, paraffin-embedded, hematoxylin and eosin-stained sections were examined to enumerate numbers of leukocytes present in the dermis. In all histological studies, examination of the slides was performed by an observer who was not aware of the identity of individual sections.

Chemerin Expression and Purification Using Baculovirus

The following carboxyl-terminal His₈-tagged proteins were expressed using baculovirus-infected insect cells as previously described (Zabel, B. A., S. J. Allen, P. Kulig, J. A. Allen, J. Cichy, T. M. Handel, and E. C. Butcher. 2005. Chemerin activation by serine proteases of the coagulation, fibrinolytic, and inflammatory cascades. J Biol Chem 280:34661-34666): “serum form” human chemerin (NH₂-ADPELTE . . . FAPHHHHHHHH-COOH) (SEQ ID NO:07), “pro-form” human chemerin (NH₂-ADPELTE . . . LPRSPHHHHHH-COOH) (SEQ ID NO:08), and “serum form” mouse chemerin (NH₂-ADPTEPE . . . FAPHHHHHHHH-COOH) (SEQ ID NO:09). Since certain experiments required non-tagged proteins, the His₈-tag was proteolytically removed by treatment with carboxypeptidase A (Sigma), generating the respective proteins NH₂-ADPELTE . . . FAPH-COOH (SEQ ID NO:10), NH₂-ADPELTE . . . RSPH-COOH (SEQ ID NO:11), and NH₂-ADPTEPE . . . FAPH-COOH (SEQ ID NO:12), where the underlined residues are non-native. The proteins were lyophilized and checked for purity using electrospray mass spectrometry.

Chemerin Binding Assays

For chemerin binding/anti-mCCRL2 mAbs displacement assays, total peritoneal exudate cells were incubated with various concentrations of chemerin or CCL2 for 5 minutes on ice in binding buffer, washed with PBS, and stained with primary antibodies (either anti-mCMKLR1BZ2E3 or IgE, +Fc block) for 45 min on ice. The cells were washed in PBS and stained with secondary anti-rat IgG PE or anti-mouse IgE PE (+goat IgG) for 30 min on ice, washed with PBS, stained with directly conjugated F4/80 and c-kit mAbs, and analyzed by flow cytometry. For radioligand binding assays, radioiodinated chemerin (residues 21-148, R&D Systems, custom radiolabeling performed by Perkin Elmer) was provided as a kind gift from Dr. Juan Jaen (ChemoCentryx, Mountain View, Calif.). The specific activity of the ¹²⁵I-labeled chemerin was 97 Ci/g. For competition binding assays, L1.2 cells transfected with huCCRL2, mCCRL2, or mCMKLR1 were plated into 96-well plates at 0.5×10⁶ cells/well. Cells were incubated in binding buffer (Hanks+0.5% BSA) for 3 hr at 4° C. shaking with 1 nM ¹²⁵I-chemerin and increasing concentrations of chemerin, His₈-tagged chemerin, or peptide (9-aa YFPGQFAFS (SEQ ID NO:13), corresponding to chemerin residues 149-157) as competitors. For saturation binding assays, mCCRL2/L1.2 cells were plated at 0.5×10⁶ cells/well. Nonspecific binding was measured in the presence of 100 nM cold chemerin. Binding was terminated by washing the cells in saline buffer, and bound radioactivity was measured. Data were analyzed using Prism (GraphPad Software). Binding data (triplicate or quadruplicate wells) were fitted to one-site binding hyperbola for saturation assays, or to a one-site competition curve for competition assays. For direct chemerin binding immunofluorescence assays, mCCRL2-HA, huCCRL2-HA, mCMKLR1-HA, mCRTH2-HA L1.2 transfectants were incubated for 30 min on ice with 10 nM His₈-tagged serum form human chemerin and the indicated concentration of untagged chemerin in binding buffer (PBS with 0.5% BSA, 0.02% azide). The cells were then washed with PBS, and incubated with anti-His₆ PE (+2% goat serum) for 30 min on ice, washed and analyzed by flow cytometry. Similar binding experiments were performed on total WT or CCRL2 KO peritoneal exudate cells with the indicated combinations and concentrations of pro-form or serum form His₈-tagged or untagged chemerin. Following chemerin binding, the cells were stained with directly conjugated F4/80 and c-kit mAbs and analyzed by flow cytometry.

In Vitro Transwell Chemotaxis

For migration experiments using cell lines, 2.5×10⁵ cells/100 μl chemotaxis media (RPMI+10% fetal calf serum) were added to the top wells of 5-um pore transwell inserts (Costar, Corning, N.Y., USA), and test samples in 600 μl media were added to the bottom wells. After incubating the transwell plates for 1.5 hours at 37° C., the bottom wells were harvested and flow cytometry was used to assess migration. For primary cell chemotaxis, 1×10⁶ cells/100 μl were added to the top well, and, following a 2 hour incubation at 37° C., polystyrene beads (15.0 μm diameter, Polysciences, Warrington, Pa., USA) were added to each well to facilitate normalization of the cell count. The cells were then stained for c-kit, F4/80, and/or CD11b expression and analyzed by flow cytometry. A ratio was generated and percent input migration was calculated.

Intracellular Calcium Mobilization

Chemoattractant-stimulated Ca²⁺-mobilization was performed following Alliance for Cell Signaling protocol ID PP00000210. Cells (3×10⁶/mL) were loaded with 4 μM Fluo4-AM and 0.16% Pluronic acid F-127 (Molecular Probes) in modified Iscove's medium (Iscove's medium with 1% heat inactivated bovine calf serum and 2 mM L-glutamine, Invitrogen) for 30 minutes at 37° C. The samples were mixed every 10 minutes during loading, washed once, resuspended at up to 2×10⁶/mL in the same buffer, and allowed to rest in the dark for 30 minutes at room temperature. Chemoattractant-stimulated change in Ca²⁺-sensitive fluorescence of Fluo-4 was measured over real-time with a FACsScan flow cytometer and CellQuest software (BD Biosciences) at room temperature under constant stirring (500 rpm). Fluorescent data were acquired continuously up to 1024 seconds at 1-second intervals. The samples were analyzed for 45 seconds to establish basal state, removed from the nozzle to add the stimuli, and then returned to the nozzle. Mean channel fluorescence over time was analyzed with FlowJo (TreeStar, Ashland, Oreg., USA) software. In some experiments, to identify mast cells, mixed peritoneal leukocytes were pre-incubated with c-kit-PerCP mAb for 3 minutes immediately before the start of each sample. In other experiments, mCCRL2HA/L1.2 or empty vector pcDNA3/L1.2 cells were loaded for 30 minutes with 1000 nM serum form chemerin (incubated in binding buffer on ice), washed 2× with PBS, and resuspended in Iscoves media at 2×10⁶/ml. 500 μl of these chemerin-loaded cells were added to mCMKLR1/L1.2 cells loaded with Fluo4-AM, and calcium mobilization was evaluated.

Receptor Internalization Assay

mCMKLR1-HA, huCCRL2-HA, and mCCRL2-HA L1.2 transfectants were incubated with for 15 minutes with 100 nM serum form chemerin at the indicated temperature in cell culture media. The cells were then washed with 200 μl PBS and stained with mouse anti-HA (Covance, Inc) or mIgG1 isotype control, followed by staining with secondary anti-mouse IgG1 PE, fixed, and analyzed by flow cytometry.

Ligand-Independent Receptor Internalization Assay

mCMKLR1-HA and mCCRL2-HA L1.2 transfectants were loaded for 30 minutes on ice with primary antibody (anti-HA or mIgG1 isotype control). The cells were washed with 200 μl PBS, incubated for the indicated times at 37° C. to allow for receptor internalization, and then placed on ice. The cells were then incubated with secondary anti-mouse IgG₁ PE, and analyzed by flow cytometry.

Chemerin Internalization Assay

mCCRL2-HA L1.2 transfectants or total peritoneal exudate cells were incubated with 10 nM His₈-tagged serum form chemerin for 30 minutes on ice. The primary cells were also stained with F4/80 and c-kit mAbs. Secondary anti-His₆ PE was added to the cells and incubated for 30 minutes on ice. The cells were then incubated for the indicated times at 37° C. to allow for chemerin internalization. The cells were incubated for 5 minutes on ice with either PBS or acid wash buffer (0.2 M acetic acid, 0.5 M NaCl), and then analyzed by flow cytometry. Mast cells were identified by gating on SSC^{high} F4/80⁻ c-kit⁺ cells.

Chemerin Sequestration Assay

2 nM serum form chemerin was incubated with 40 million cells of the indicated transfectant lines (or media alone) for 15 minutes at 37° C. The cells were removed by centrifugation, and a volume of the conditioned media equivalent to 0.2 nM chemerin (barring any sequestration or degradation) was tested in transwell chemotaxis using mCMKLR1-HA/L1.2 cells.

Statistics

The unpaired Student's t-test (2-tailed), Mann Whitney U-test (2-tailed), or ANOVA was used for statistical evaluation of the results, as indicated.

FITC-Induced Contact Hypersensitivity (CHS)

FITC-induced CHS was performed as described previously (Suto, H., S. Nakae, M. Kakurai, J. D. Sedgwick, M. Tsai, and S. J. Galli. 2006. Mast cell-associated TNF promotes dendritic cell migration. J Immunol 176:4102-4112) with minor modifications. Mice were shaved on abdomen 2 days before FITC-sensitization. Mice were then treated with 200 μl of 2.0% (w/v) FITC isomer I (SIGMA) suspension in acetone-dibutyl phthalate (1:1). Five days after sensitization with 2.0% FITC, mice were challenged with 40 μl of vehicle alone to the right ear (20 μl to each side) and 0.5% (w/v) FITC solution to the left ear (20 μl to each side). Each mouse was housed in a separate cage to prevent contact with each other after FITC challenge. Ear thickness was measured before and at multiple intervals after FITC challenge with an engineer's microcaliper (Ozaki MFG. CO., LTD., Itabashi, Tokyo, Japan).

RNA Expression Analysis

A RNA dot blot array was purchased from BD Clontech and hybridizations were performed according to the manufacturer's recommendation. A full-length gel-purified mCCRL2 cDNA probe was radiolabeled with ³²P using RediPrime reagents (Amersham Biosciences) according to manufacturer's specifications.

### Example 1

### mCCRL2-Specific mAbs Selectively Stain Mouse Mast Cells

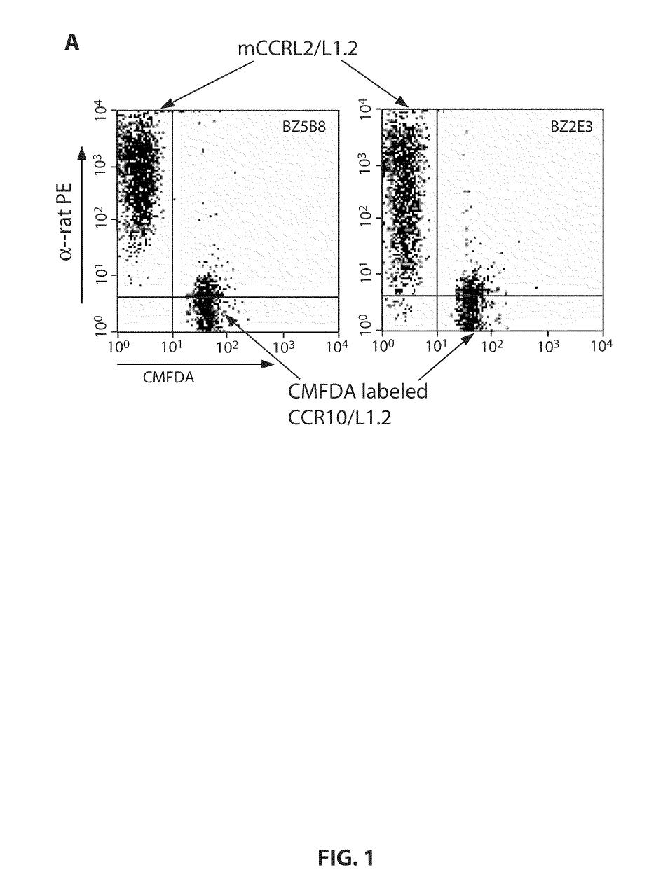

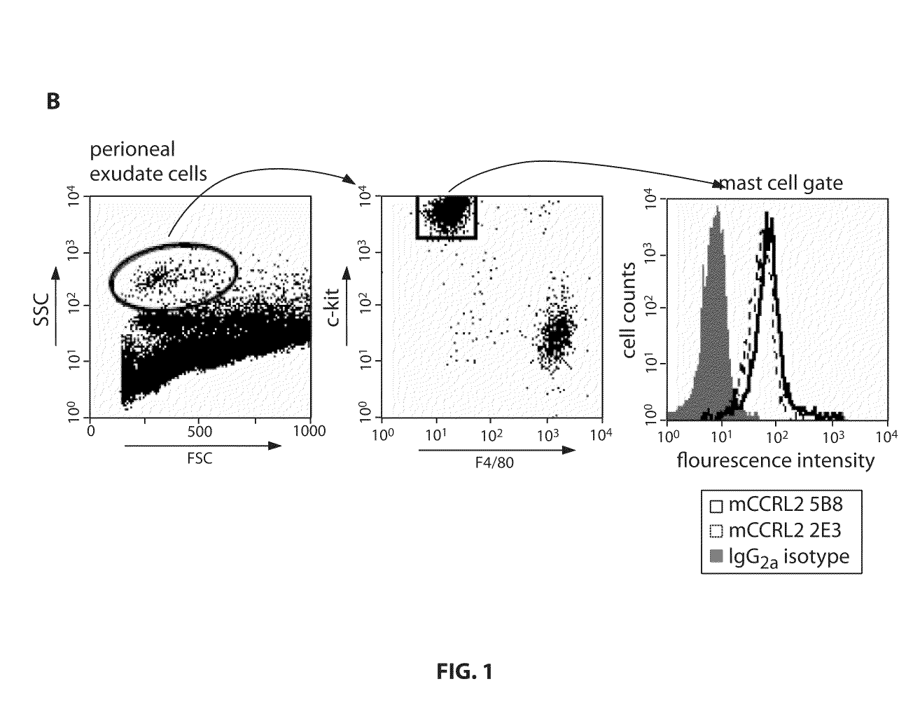

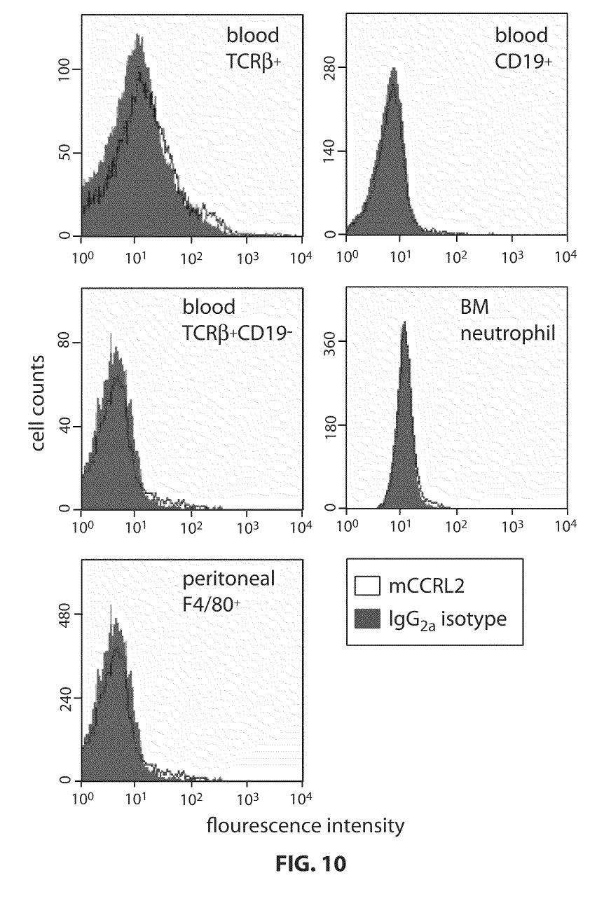

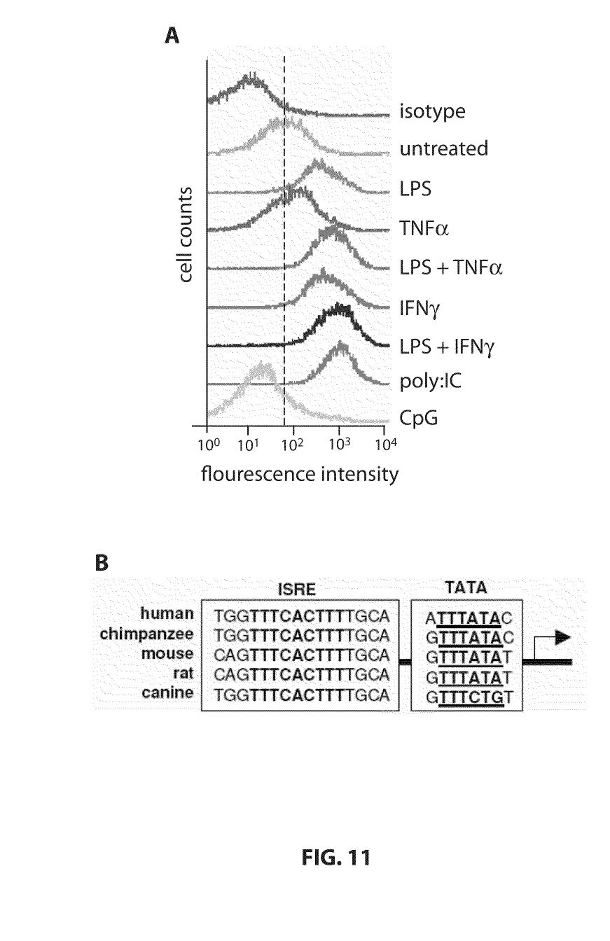

We generated monoclonal antibodies BZ5B8 and BZ2E3 (IgG₂ₐK) with reactivity to the extracellular amino-terminal domain of mCCRL2 (FIG. 1, Panel A). The antibodies were specific to mCCRL2-HA/L1.2 transfectants, displaying no cross-reactivity with other GPCR/L1.2 transfectants tested (mCMKLR1, huCMKLR1, mCRTH2, huCCRL2, or mCCR10). Reactivity with CXCR1-through-6 and CCR1-10 was excluded by lack of staining of blood cell subsets or cultured mouse cells known to express these receptors (FIG. 10). In agreement with published RNA expression data (Shimada, T., M. Matsumoto, Y. Tatsumi, A. Kanamaru, and S. Akira. 1998. A novel lipopolysaccharide inducible C—C chemokine receptor related gene in murine macrophages. FEBS Lett 425:490-494), peritoneal macrophages treated with LPS upregulated mCCRL2 protein expression; expression of mCCRL2 was also upregulated in such cells by treatment with TNFα, IFNγ, or poly:IC (FIG. 11, Panel A).

Freshly isolated mouse blood T cells, B cells, NK cells, bone marrow neutrophils, and resting peritoneal macrophages were all negative for mCCRL2 expression (FIG. 10). A small population of highly granular (SSC^{high}), F4/80⁻ c-kit⁺ leukocytes in the peritoneal cavity, however, uniformly stained for mCCRL2 (FIG. 1, Panel B). These cells also expressed the high affinity IgE Fc receptor FcεRI. On staining with Wright-Giemsa stain, sorted F480⁻CCRL2⁺ cells displayed intense metachromatic staining of abundant cytoplasmic granules, as did mast cells sorted as F4/80⁻ c-kit⁺ cells (FIG. 1, Panel C). Thus both traditional morphologic and immunophenotypic analyses indicate that mCCRL2 is constitutively expressed by mast cells, and the expression is surprisingly selective for mast cells in the absence of pathologic stimuli.

Mast cells derived from bone marrow progenitors in vitro (BMCMCs) upregulated expression of mCCRL2 over time in culture. Early mast cell progenitors were negative for mCCRL2, but after >2 months in culture the cells uniformly expressed detectable levels of mCCRL2, albeit the levels were lower than those on peritoneal mast cells in vivo (FIG. 1, Panel D). We confirmed RNA expression of mCCRL2 in peritoneal mast cells by real time quantitative RT-PCR (FIG. 1, Panel E).

### Example 2

### CCRL2 and Mast Cell Phenotype and Function

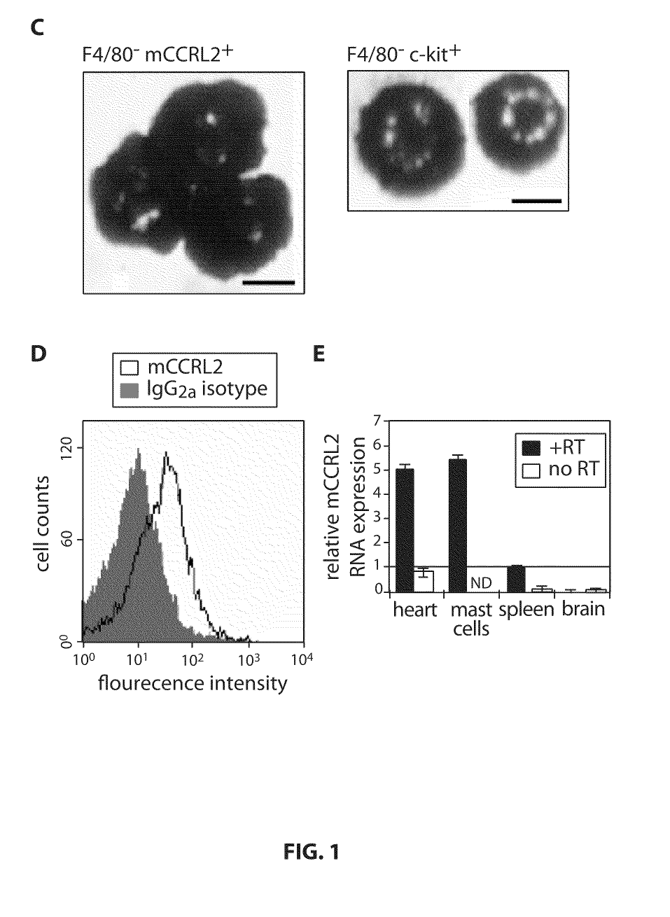

We evaluated numbers of mast cells in CCRL2 KO mice in vivo, as well as certain basic functions of CCRL2 KO BMCMC in vitro. Our anti-mCCRL2 mAbs failed to stain peritoneal mast cells from CCRL2 KO mice, confirming the genetic ablation of the gene (FIG. 2, Panel A). The mice are fertile, reproduce with the expected Mendelian distribution of KO:heterozygotes:WT and male:female ratios, and display no differences in basal mast cell numbers in the ear skin or in mesenteric windows (FIG. 2, Panel B).

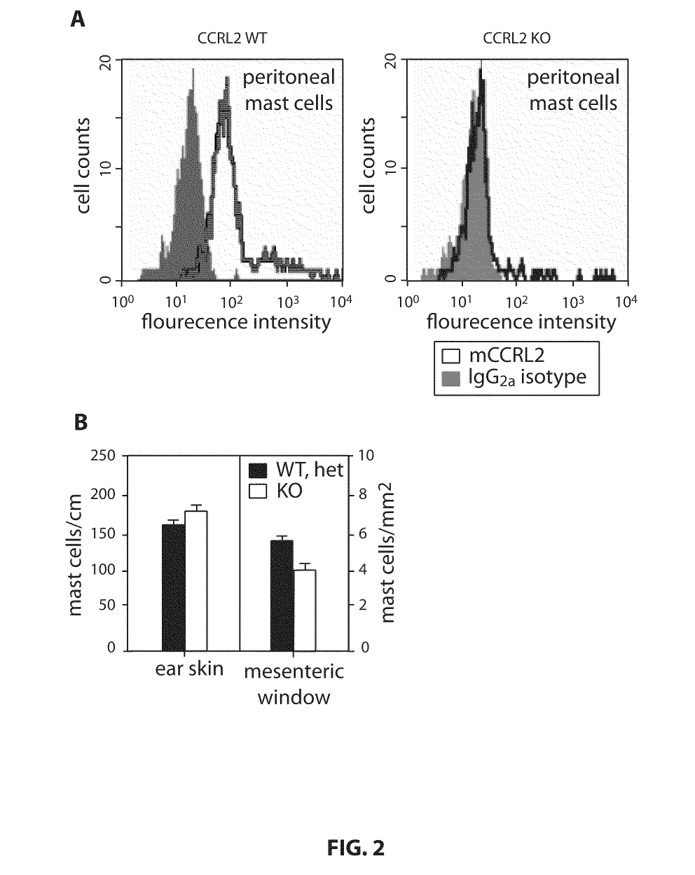

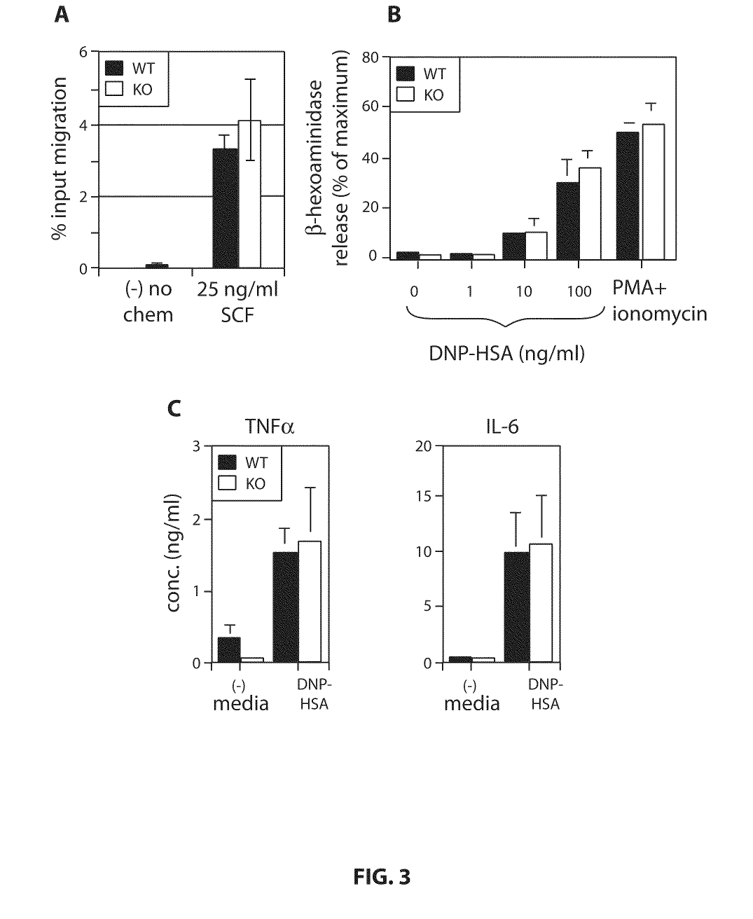

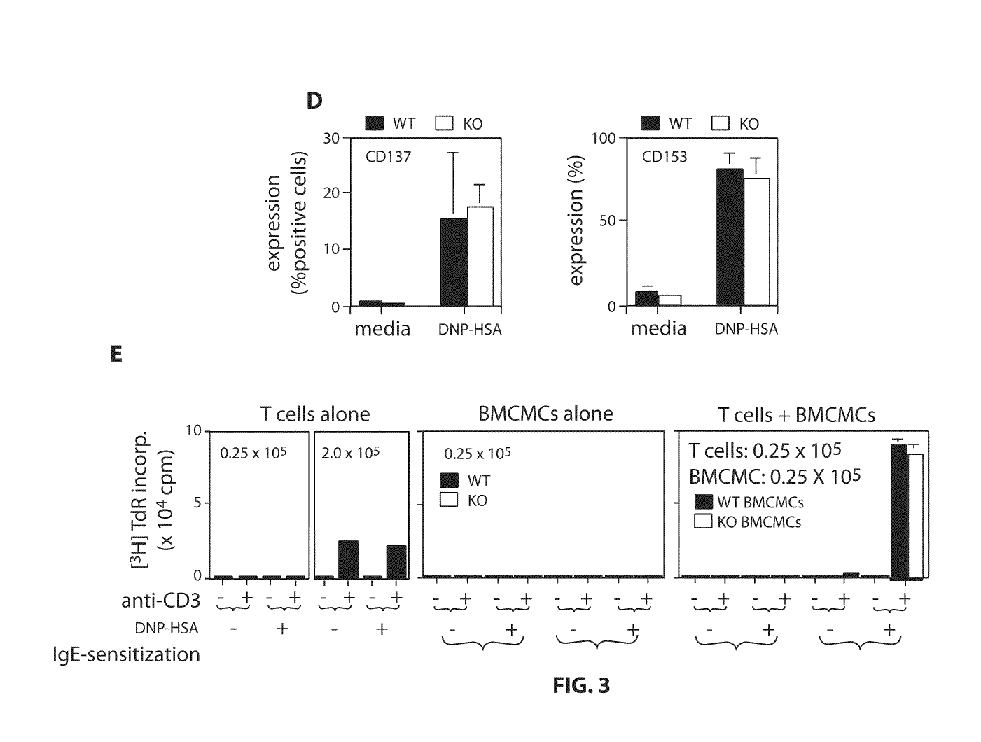

CCRL2 KO and WT BMCMCs expressed similar levels of c-kit (CD117) and FcεRI. BMCMCs from WT or mCCRL2 KO mice also displayed similar chemotactic responses to stem cell factor (SCF), indicating no inherent differences in cell migration (FIG. 3, Panel A); similar degranulation responses to antigen-mediated IgE/FcεRI crosslinking as assessed by β-hexosaminidase release (FIG. 3, Panel B); and similar activation-dependent secretion of cytokines TNFα and IL-6 (FIG. 3, Panel C). We recently showed that antigen-mediated IgE/FcεRI crosslinking upregulated expression of several co-stimulatory molecules on BMCMCs (Nakae, S., H. Suto, M. Iikura, M. Kakurai, J. D. Sedgwick, M. Tsai, and S. J. Galli. 2006. Mast cells enhance T cell activation: importance of mast cell costimulatory molecules and secreted TNF. J Immunol 176:2238-2248), however, we did not detect any CCRL2-dependent differences in CD137 (4-1BB) or CD153 (CD30L) induction (FIG. 3, Panel D). BMCMCs stimulated by antigen-mediated IgE/FcεRI crosslinking also can enhance T cell proliferation (Nakae, S., H. Suto, M. Iikura, M. Kakurai, J. D. Sedgwick, M. Tsai, and S. J. Galli. 2006. Mast cells enhance T cell activation: importance of mast cell costimulatory molecules and secreted TNF. J Immunol 176:2238-2248; Nakae, S., H. Suto, M. Kakurai, J. D. Sedgwick, M. Tsai, and S. J. Galli. 2005. Mast cells enhance T cell activation: Importance of mast cell-derived TNF. Proc Nail Acad Sci USA 102:6467-6472). While naive T cells proliferated markedly in response to treatment with anti-CD3 and co-incubation with mitomycin C-treated, antigen-specific IgE stimulated BMCMCs, there were no CCRL2-dependent differences in the ability of BMCMCs to enhance T cell proliferation (FIG. 3, Panel E) or T cell secretion of IFNγ or IL-17 in the conditions tested. Thus, the presence of absence of CCRL2 on BMCMCs did not significantly influence the basic mast cell functions tested here.

### Example 3

### Mast Cell-Expressed CCRL2 is Required for Optimal Induction of IgE-Dependent Passive Cutaneous Anaphylaxis

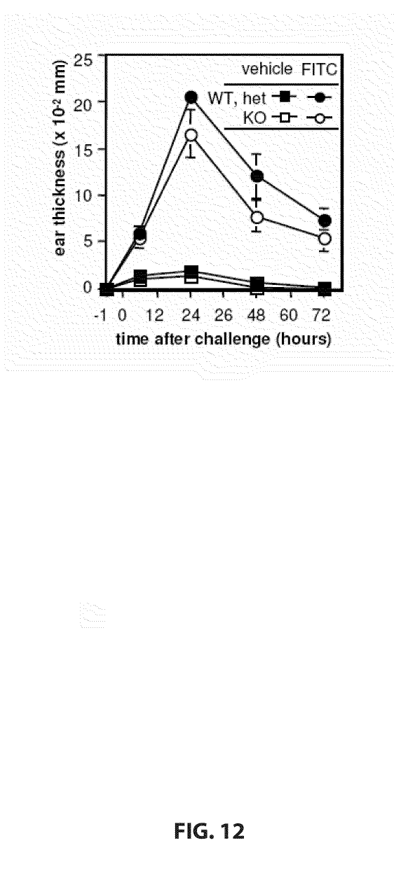

To search for potential contributions of CCRL2 to pathophysiologic responses in vivo, we next examined certain inflammatory conditions that are known to involve mast cells. Mast cells are required for optimal expression of the T cell-mediated contact hypersensitivity (CHS) induced by a protocol employing FITC (fluorescein isothiocyanate), but not that induced by other protocols employing DNFB (2,4-dinitro-1-fluorobenzene) (Takeshita, K., T. Yamasaki, S. Akira, F. Gantner, and K. B. Bacon. 2004. Essential role of MHC II-independent CD4+ T cells, IL-4 and STATE in contact hypersensitivity induced by fluorescein isothiocyanate in the mouse. Int Immunol 16:685-695; Suto, H., S, Nakae, M. Kakurai, J. D. Sedgwick, M. Tsai, and S. J. Galli. 2006. Mast cell-associated TNF promotes dendritic cell migration. J Immunol 176:4102-4112). However, CCRL2 was largely dispensable for the tissue swelling associated with FITC-triggered CHS, as both WT and CCRL2 KO mice developed statistically indistinguishable responses (FIG. 12).

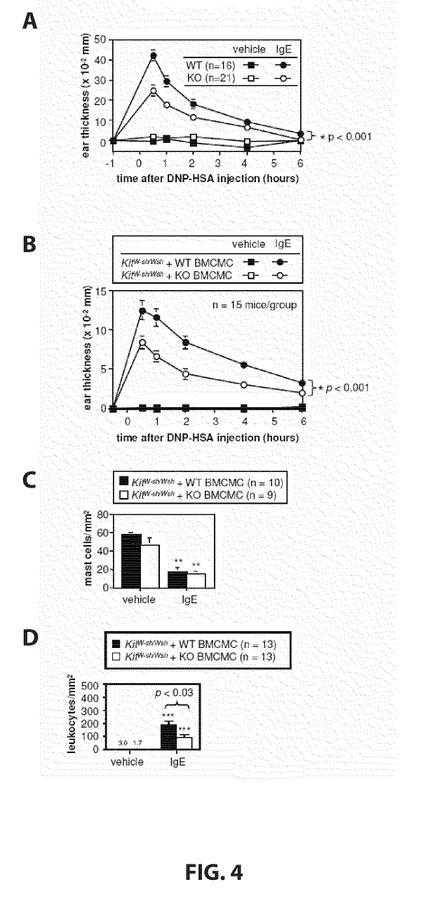

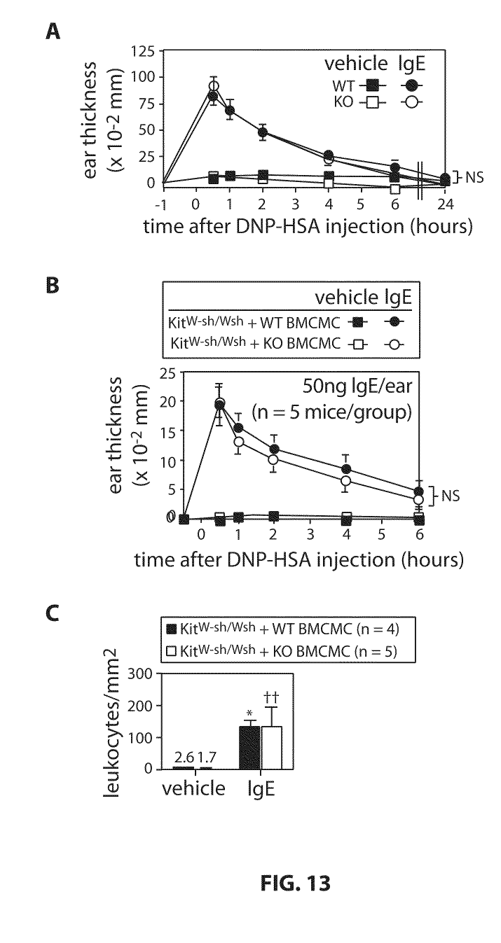

We next examined a mast cell-dependent model of atopic allergy, the IgE-dependent passive cutaneous anaphylaxis (PCA) reaction Animals sensitized with 150 ng/ear DNP-specific IgE and challenged with antigen (DNP-HSA) i.v. developed strong local inflammatory responses, with no significant difference in the tissue swelling observed in WT vs. CCRL2 KO mice (82±9 vs. 91±9×10⁻² mm of swelling at 30 min after antigen challenge, respectively [p>0.05, by Student's t-test] (FIG. 13, Panel A)). However, when the sensitizing dose of DNP-specific IgE was reduced to 50 ng/ear, the PCA reactions in CCRL2 KO mice were significantly impaired compared to those in WT mice (42.2±2.8 vs. 24.9±2.7×10⁻² mm of swelling at 30 min after antigen challenge, respectively [p<0.005, by Student's t-test] (FIG. 4, Panel A)).

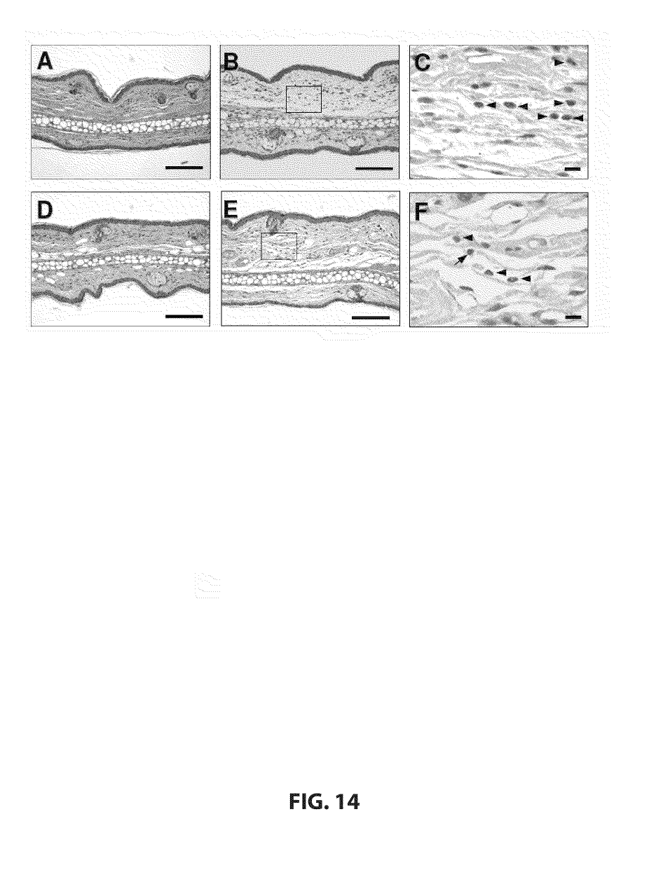

To assess the extent to which mCCRL2 expression specifically on mast cells was critical for the defect in IgE-dependent PCA observed in mCCRL2 KO mice, we engrafted mast cell-deficient Kit^{W-sh/Wsh} mice intra-dermally in the ear pinnae with either WT or mCCRL2 KO BMCMCs; 6-8 weeks later, the animals were subjected to IgE-dependent PCA. Such mast cell engraftment of mast cell-deficient Kit^{W-sh/Wsh} or Kit^{W/W-v} mice is routinely used to identify the roles of mast cells in biological responses in vivo (Galli, S. J., S, Nakae, and M. Tsai. 2005. Mast cells in the development of adaptive immune responses. Nat Immunol 6:135-142). There was no difference in the extent of PCA-associated ear swelling between Kit^{W-sh/Wsh} mice that had been engrafted with WT vs. mCCRL2 KO BMCMCs when the animals were sensitized with 50 ng/ear DNP-specific IgE and challenged with i. v. antigen (19.5±3.6 vs. 19.9±2.6×10⁻² mm of swelling at 30 min after antigen challenge, respectively [p>0.05, by Student's t-test] (FIG. 13, Panel B)). Nor was there a significant difference in the numbers of leukocytes infiltrating the dermis at these sites at 6 h after antigen challenge (FIG. 13, Panel C and FIG. 14).

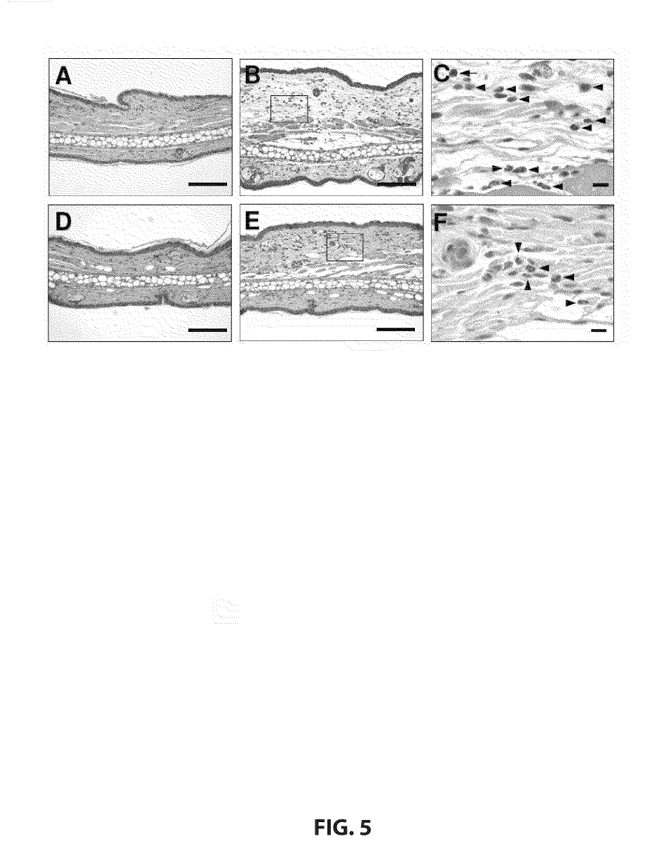

However, when the sensitizing dose of DNP-specific IgE was reduced to 5 ng/ear, there was a significant reduction in ear swelling responses in mice that had been engrafted with mCCRL2 KO BMCMCs compared with those that had been engrafted with WT BMCMCs (12.5±1.2 vs. 8.4±0.8×10⁻² mm of swelling at 30 min after antigen challenge, respectively [p<0.01, by Student's t-test] (FIG. 4, Panel B). There were no significant differences in the total number of mast cells detected histologically in WT vs. CCRL2 KO BMCMC-engrafted ears, thus ruling out any CCRL2-dependent effects on mast cell engraftment efficiency (FIG. 4, Panel C). However, at 6 h after antigen challenge, IgE-dependent PCA reactions in ears that had been engrafted with CCRL2 KO mast cells contained ˜50% fewer leukocytes (predominantly neutrophils and mononuclear cells) than did reactions in ears that had been engrafted with wild type mast cells [p<0.03 by the Mann Whitney U-test] (FIG. 4, Panel D and FIG. 5). IgE-dependent PCA reactions were associated with a marked reduction in the numbers of dermal mast cells which could be identified in histological sections of these sites based on the staining of the cells' cytoplasmic granules (FIG. 4, Panel C), an effect that most likely reflected extensive mast cell degranulation at these sites (Wershil, B. K., T. Murakami, and S. J. Galli. 1988. Mast cell-dependent amplification of an immunologically nonspecific inflammatory response. Mast cells are required for the full expression of cutaneous acute inflammation induced by phorbol 12-myristate 13-acetate. J Immunol 140:2356-2360; Martin, T. R., T. Takeishi, H. R. Katz, K. F. Austen, J. M. Drazen, and S. J. Galli. 1993. Mast cell activation enhances airway responsiveness to methacholine in the mouse. J Clin Invest 91:1176-1182).

We conclude that while mast cell-expressed mCCRL2 is not required for the development of IgE-dependent PCA reactions in vivo, mast cell expression of CCRL2 can significantly enhance the local tissue swelling and leukocyte infiltrates associated with such reactions in mice that have been sensitized with relatively low amounts of antigen-specific IgE.

### Example 4

### CCRL2 Binds Chemerin

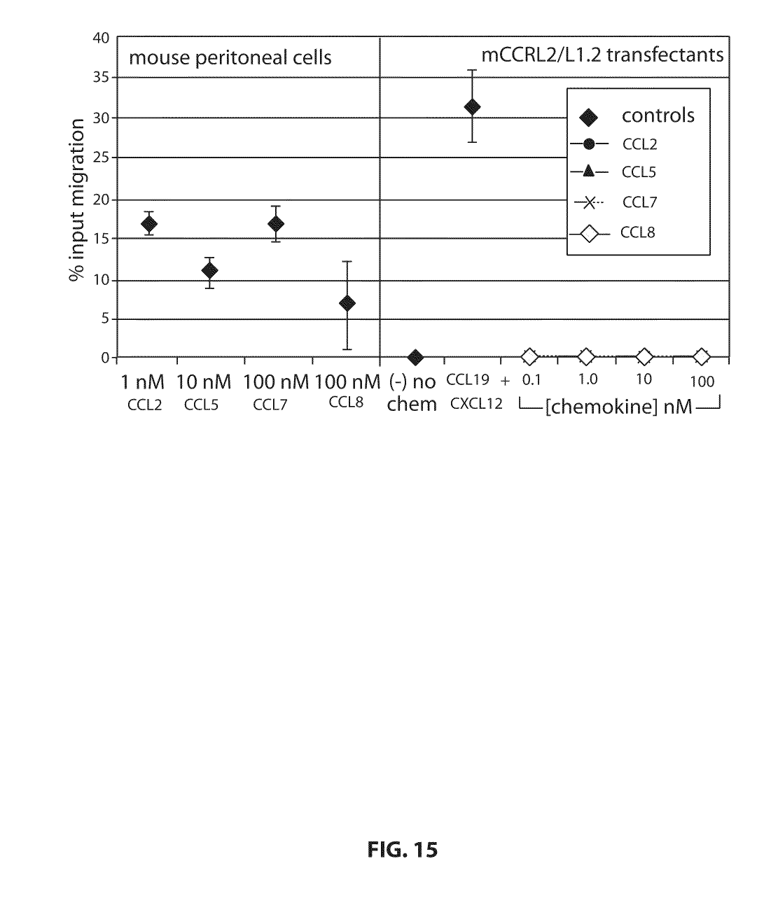

To investigate possible functional roles for CCRL2, we set out to validate/identify CCRL2 ligands. It was reported that mCCRL2/HEK293 transfectants respond functionally to CCR2 ligands CCL2, CCL5, CCL7, and CCL8 via intracellular calcium mobilization and transwell chemotaxis (Biber, K., M. W. Zuurman, H. Homan, and H. W. Boddeke. 2003. Expression of L-CCR in HEK 293 cells reveals functional responses to CCL2, CCL5, CCL7, and CCL8. J Leukoc Biol 74:243-251), although this conclusion is controversial (Galligan, C. L., W. Matsuyama, A. Matsukawa, H. Mizuta, D. R. Hodge, O. M. Howard, and T. Yoshimura. 2004. Up-regulated expression and activation of the orphan chemokine receptor, CCRL2, in rheumatoid arthritis. Arthritis Rheum 50:1806-1814; Mantovani, A., R. Bonecchi, and M. Locati. 2006. Tuning inflammation and immunity by chemokine sequestration: decoys and more. Nat Rev Immunol 6:907-918). These chemokines did not induce migration of mCCRL2/L1.2 transfectants in our in vitro transwell chemotaxis assays (FIG. 15). We also tested a panel of known chemoattractants (CCL11, CCL17, CCL22, CCL25, CCL27, CCL28, CXCL9, and CXCL13), as well as protein extracts from homogenized mouse tissues (lungs, kidney, liver, brain, and spleen) and found that none stimulated mCCRL2-dependent chemotaxis in our in vitro transwell chemotaxis assays. Given the aberrant “DRYLAIV” motif present in mouse and human CCRL2, we and others postulated that mCCRL2 may act as a “silent” receptor (Mantovani, A., R. Bonecchi, and M. Locati. 2006. Tuning inflammation and immunity by chemokine sequestration: decoys and more. Nat Rev Immunol 6:907-918), capable of binding chemoattractant(s) but incapable of transducing signals via classical second messengers. That hypothesis is consistent with the negative results obtained in our efforts to induce chemotaxis of mCCRL2/L1.2 transfectants in our in vitro transwell chemotaxis assays.

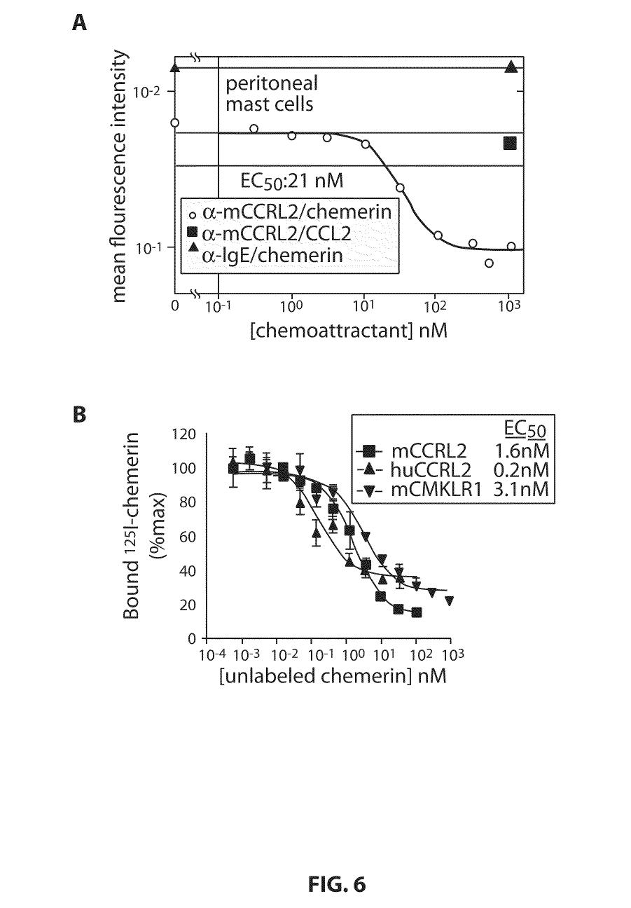

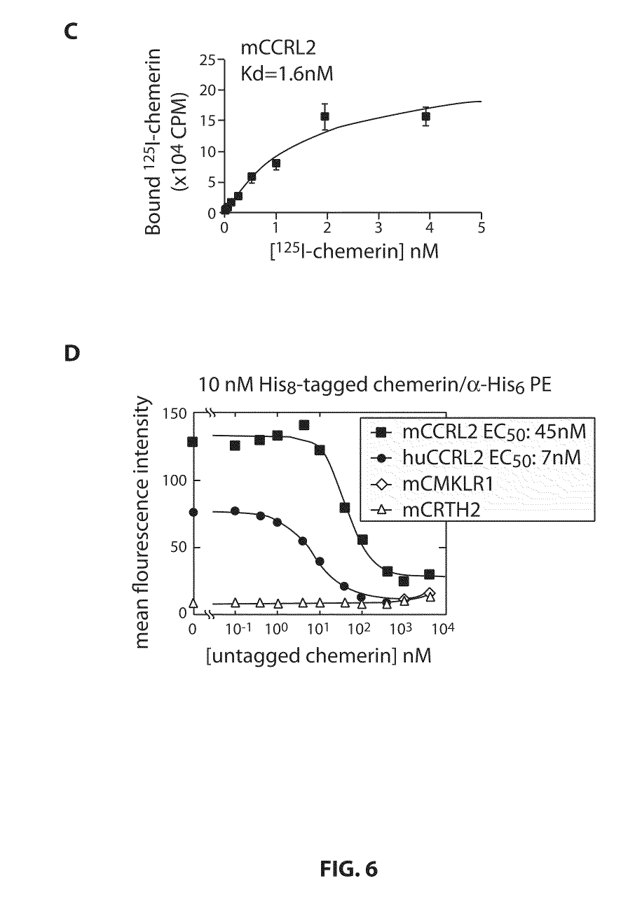

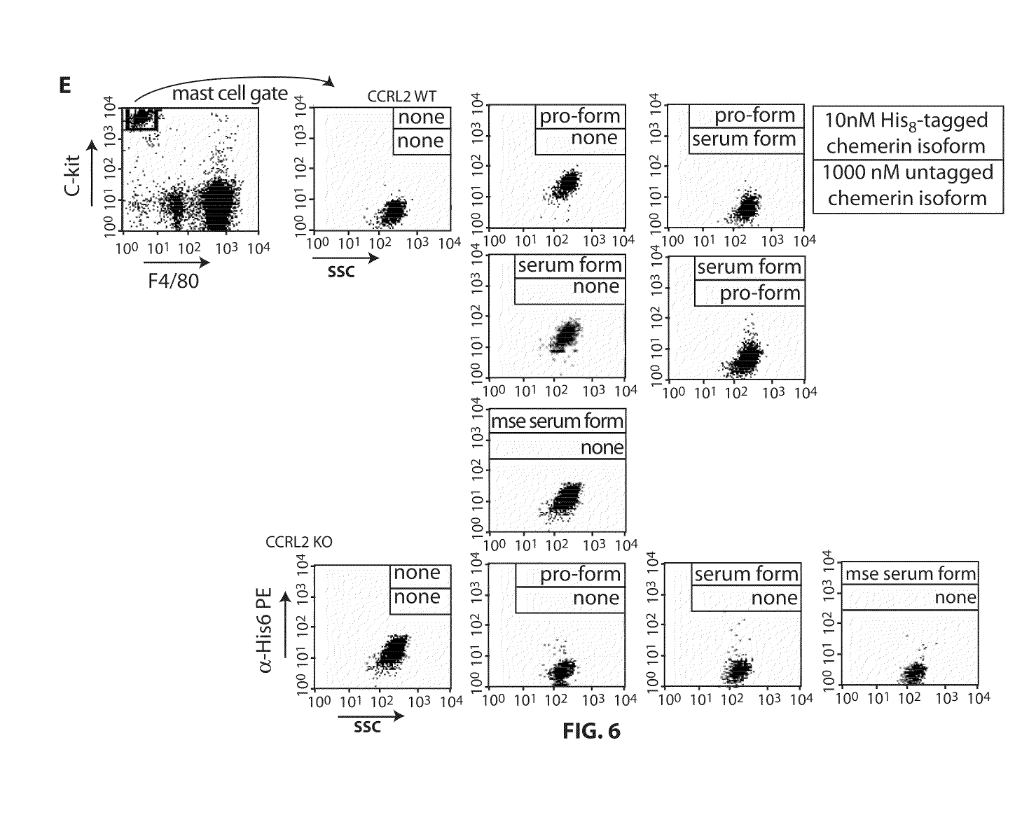

Although we failed to identify evidence of signaling effects of any of the tested chemoattractants, we were able to identify a high affinity ligand for the receptor: in independent studies in which we were using our anti-CCRL2 mAbs as controls for staining, we serendipitously discovered that chemerin, a protein ligand for signaling receptor CMKLR1 (chemokine-like receptor 1, reviewed in (Zabel, B. A., L. Zuniga, T. Ohyama, S. J. Allen, J. Cichy, T. M. Handel, and E. C. Butcher. 2006. Chemoattractants, extracellular proteases, and the integrated host defense response. Exp Hematol 34:1021-1032)), inhibited the binding of mCCRL2-specific mAbs to mouse peritoneal mast cells. In FIG. 6, Panel A we illustrate the potent ability of chemerin to inhibit anti-CCRL2 staining of mouse peritoneal mast cells. Increasing concentrations of chemerin blocked the binding of anti-mCCRL2 BZ5B8 (FIG. 6, Panel A) and BZ2E3 (data not shown) in a dose-dependent manner (EC₅₀=21 nM). The effect was specific to anti-mCCRL2:mCCRL2 interactions, since binding of IgE to FcRεI was unaffected by 1000 nM chemerin (FIG. 6, Panel A); and 1000 nM CCL2 had no effect on CCRL2 staining (FIG. 6, Panel A).

To confirm the identification of CCRL2 as a chemerin receptor, we performed radioligand-binding studies using iodinated chemerin. Cells were incubated with a fixed concentration of radiolabeled human chemerin plus increasing concentrations of unlabelled chemerin. Chemerin bound specifically to mCCRL2-HA/L1.2 transfectants (EC₅₀=1.6 nM) (FIG. 6, Panel B), but no binding was detected to untransfected or mCRTH2-HA-transfected cells (a prostaglandin D₂-binding chemoattractant receptor, data not shown). Furthermore, despite being the most divergent mouse-to-man orthologs in the chemoattractant receptor subfamily, huCCRL2 also bound specifically to chemerin (EC₅₀=0.2 nM) (FIG. 6, Panel B). The binding affinity of chemerin for CCRL2 was similar to if not slightly better than chemerin binding to the first identified chemerin receptor, mCMKLR1 (EC₅₀=3.1 nM) (FIG. 6, Panel B). In saturation-binding studies, chemerin bound to mCCRL2 at a single binding site with a calculated Kd of 1.6 nM (FIG. 6, Panel C).

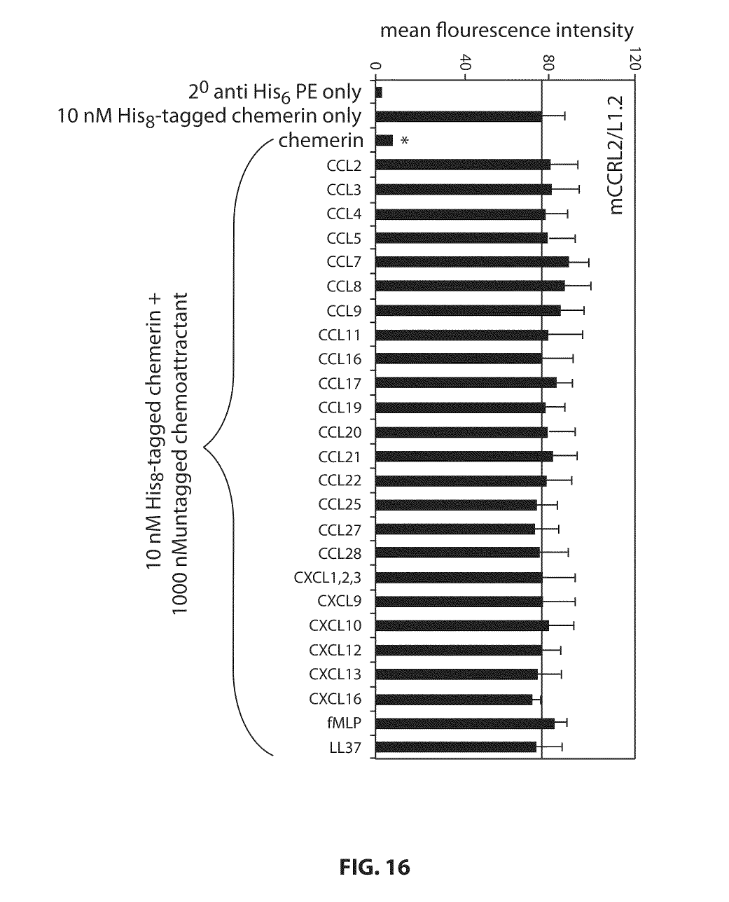

We developed an immunofluorescence-based chemerin-binding assay to evaluate chemerin binding by flow cytometry. Cells were incubated with a fixed concentration of C-terminal His₈-tagged serum form human chemerin plus increasing concentrations of untagged chemerin, and anti-His₆ PE was used to detect binding. In this assay, chemerin bound specifically to mCCRL2-HA (EC₅₀=45 nM) and huCCRL2 (EC₅₀=7 nM) L1.2 transfectants (FIG. 6, Panel D). Chemerin binding to CCRL2 was not affected by a variety of other chemoattractants (FIG. 16), and mCRTH2-HA/L1.2 transfectants did not bind to chemerin (FIG. 6, Panel D), demonstrating specificity for chemerin:CCRL2 interactions. Interestingly, we were unable to detect chemerin binding to mCMKLR1-HA/L1.2 transfectants in the immunofluorescence chemerin-binding assay (FIG. 6, Panel D): this may reflect inhibition of binding by the C-terminal His₈ tag (which would be analogous to the inhibitory activity of the carboxyl-terminal residues in the chemerin pro-form); or potentially inaccessibility of the His₈ epitope to the detection mAbs when His₈-tagged chemerin is bound to CMKLR1.

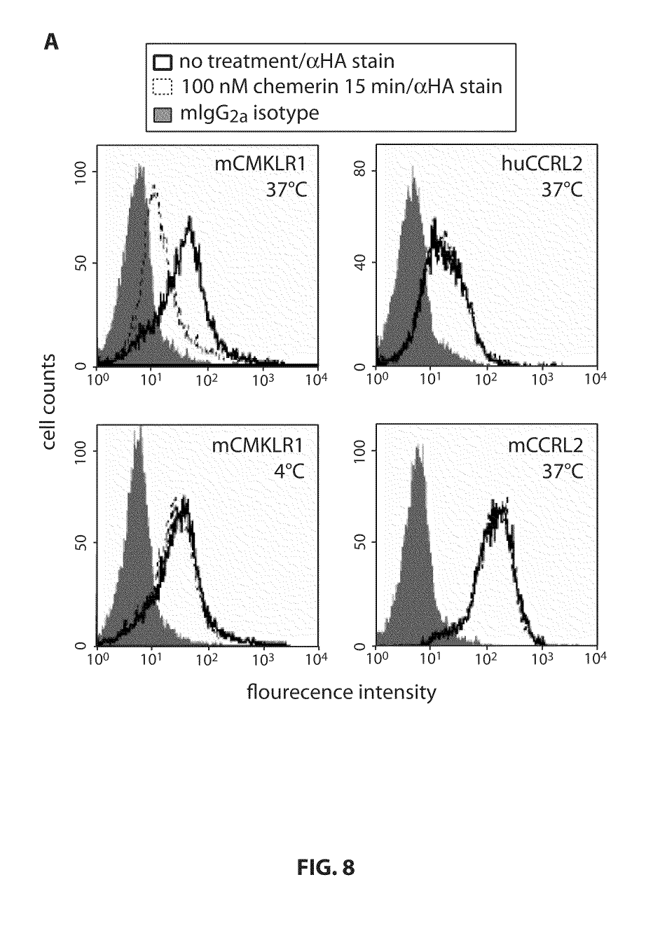

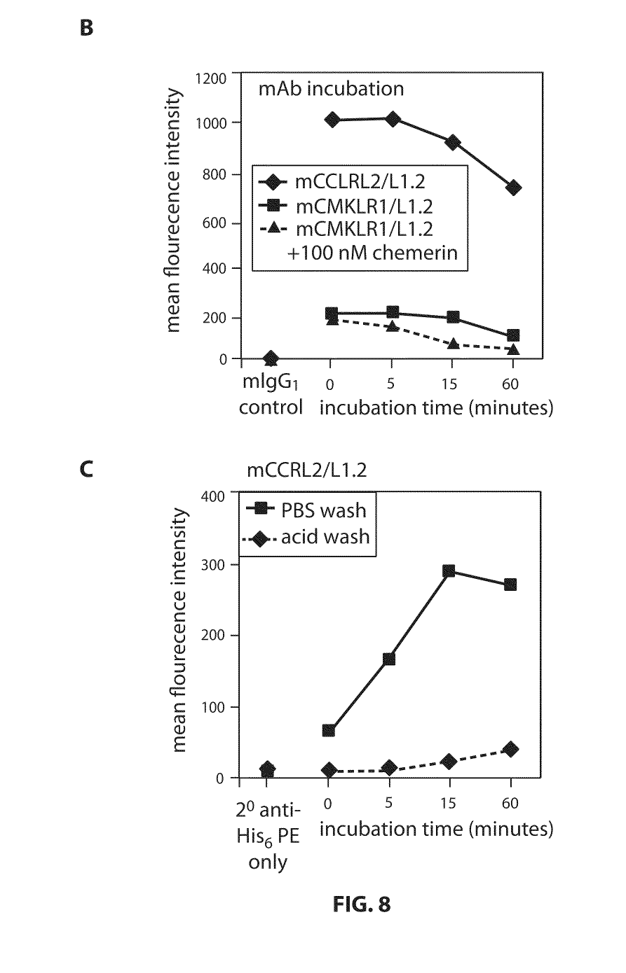

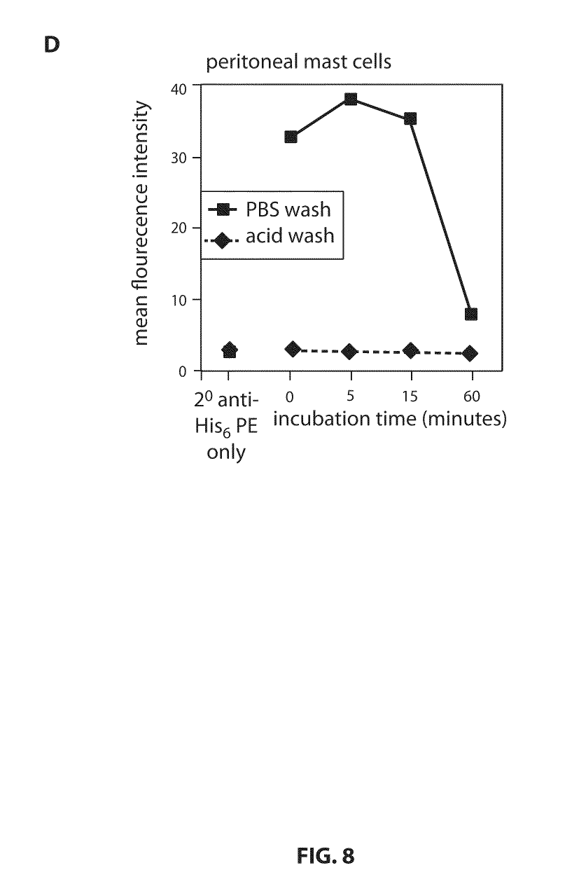

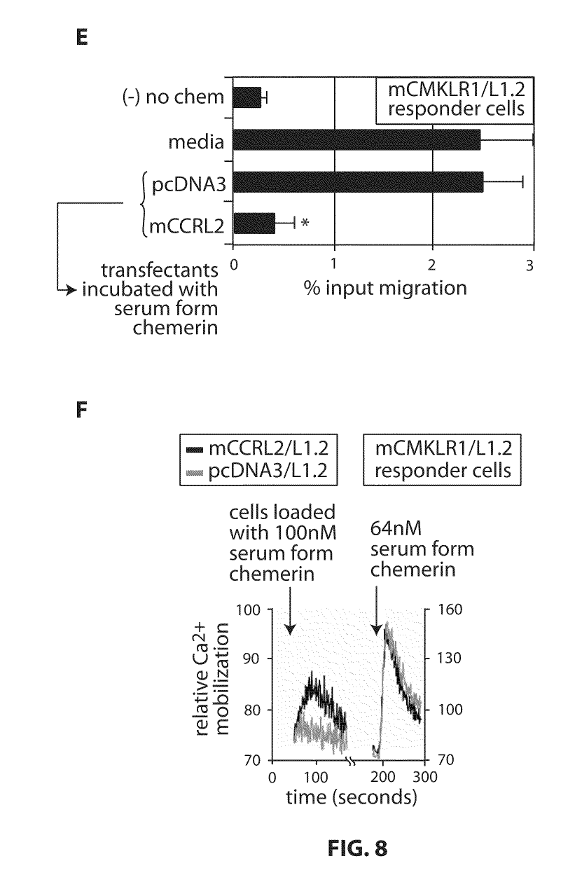

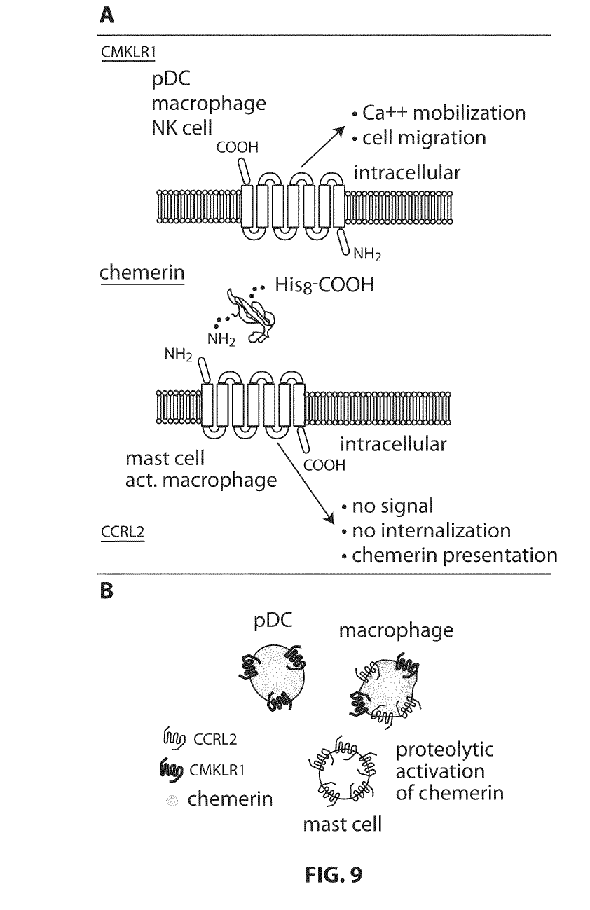

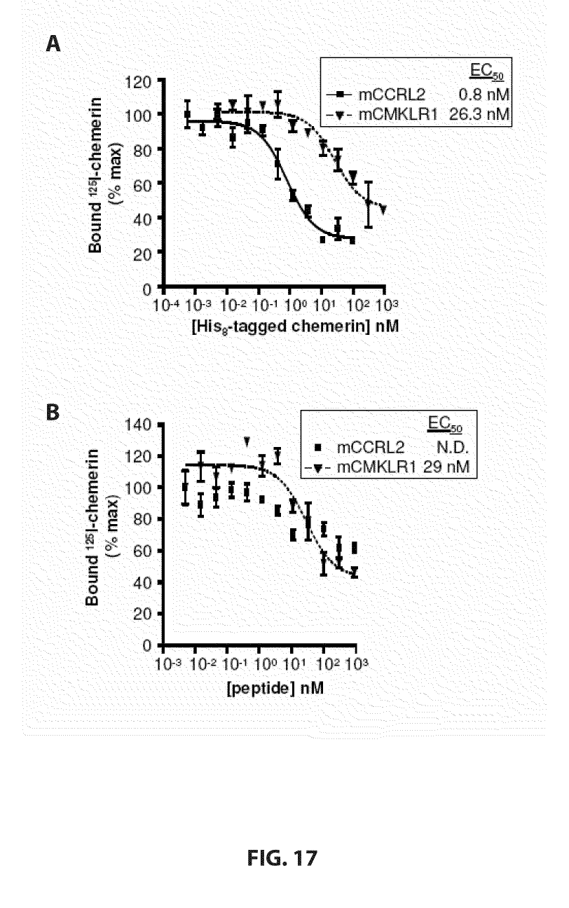

In radioligand binding studies, the His₈-tag had little effect on the potency of chemerin binding to mCCRL2 (EC₅₀=0.8 nM); however, His₈-tagged chemerin bound with 10-fold less potency to mCMKLR1 (EC₅₀=26.3 nM) (FIG. 17, Panel A). The bioactive 9-mer carboxyl-terminal chemerin peptide (residues 149-157, YFPGQFAFS) was 10-fold less potent (EC₅₀=26.2 nM, FIG. 8, Panel A) than chemerin protein in binding to CMKLR1. In CCRL2 binding, however, the bioactive chemerin peptide wan an inefficient competitor (EC₅₀ could not be determined, FIG. 17, Panel B).

Thus, the data indicate distinct binding modes for chemerin and CCRL2 vs. chemerin and CMKLR1: the carboxyl-terminal domain of chemerin that is critical for binding to CMKLR1 is relatively uninvolved and unencumbered when chemerin is bound to CCRL2.

Freshly isolated mouse peritoneal mast cells also bound to chemerin (FIG. 6, Panel E); and there was no obvious preference for binding of the pro-form vs. the active serum form (this was also observed in radioligand binding studies using L1.2 transfectants, data not shown). Moreover, mouse peritoneal mast cells bound both human and mouse chemerin (FIG. 6, Panel E). Finally, peritoneal mast cells from mCCRL2 KO mice did not bind to chemerin, further confirming the role of CCRL2 in the binding of chemerin to such mast cells (FIG. 6, Panel E).

### Example 5

### Chemerin does not Trigger CCRL2-Mediated Cell Migration or Intracellular Calcium Mobilization

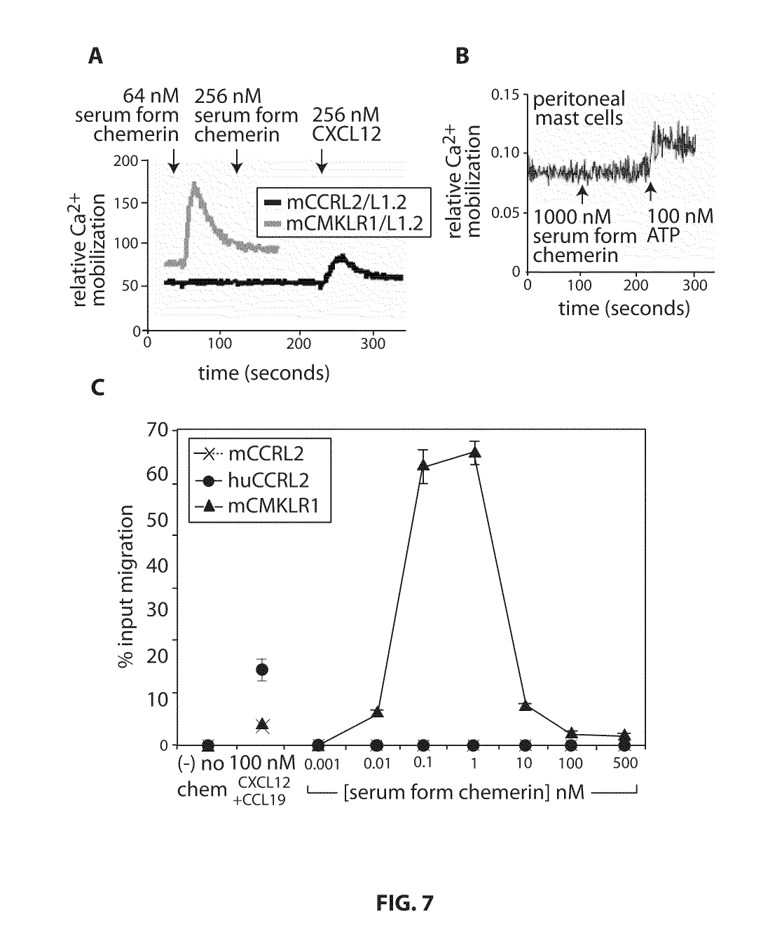

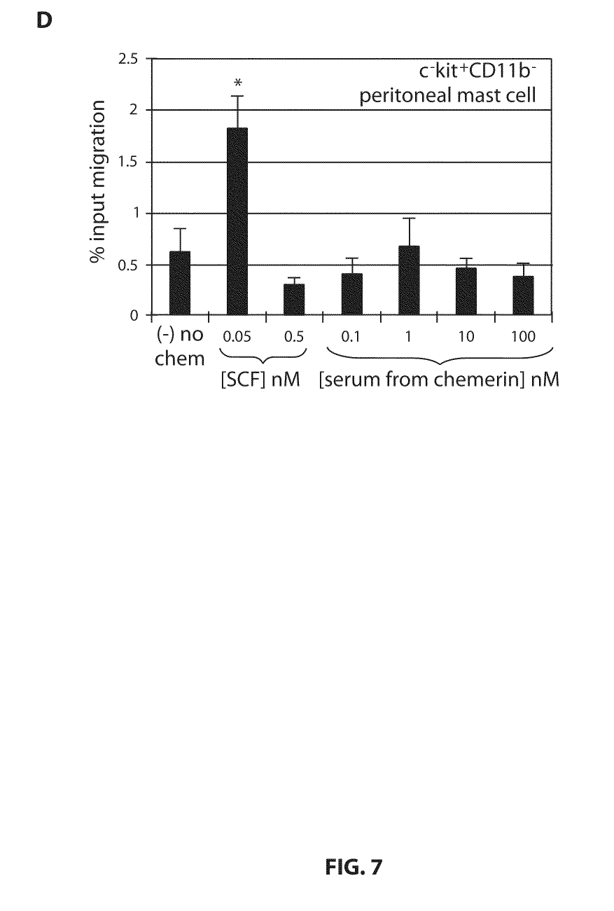

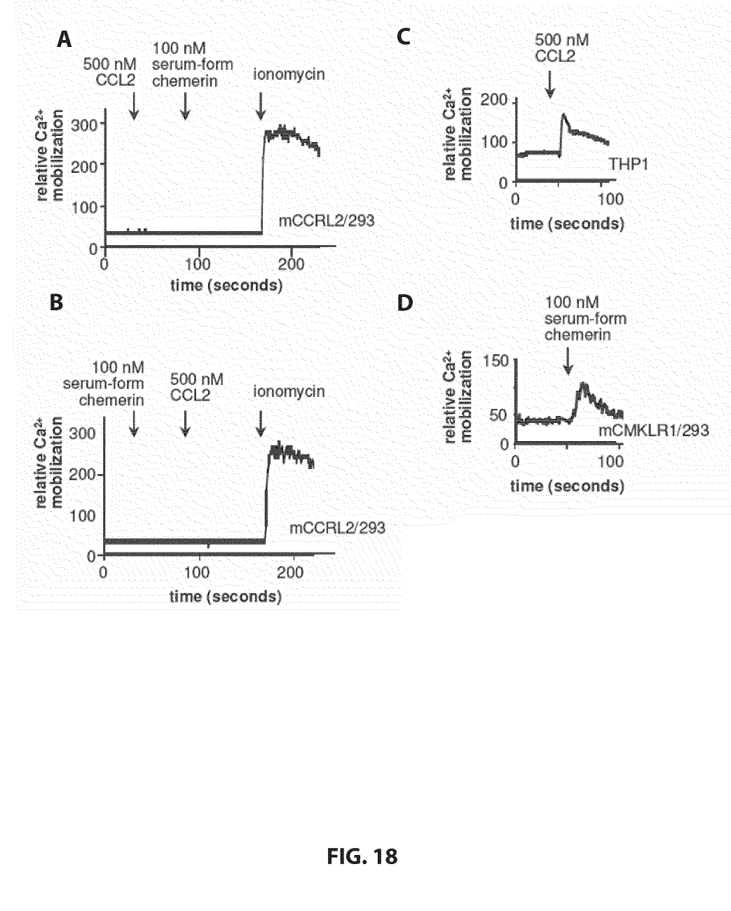

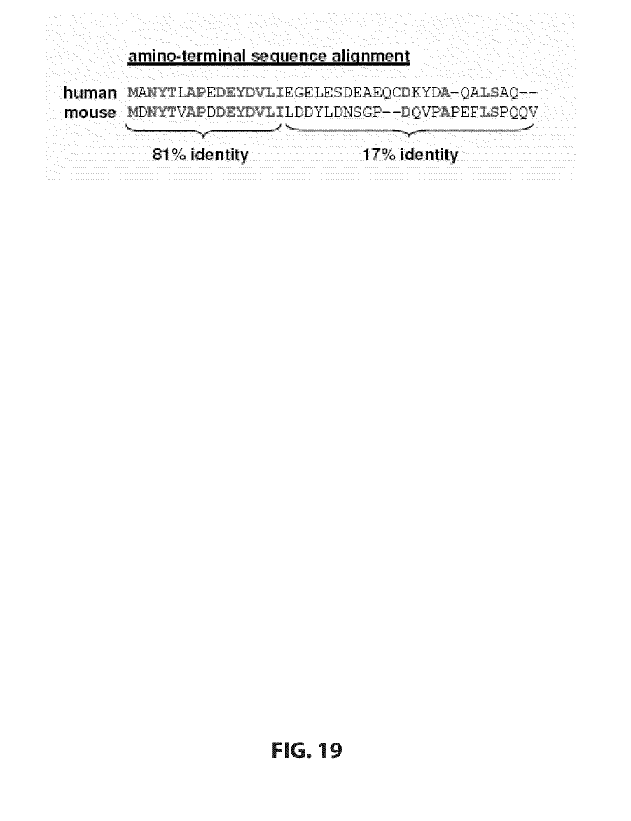

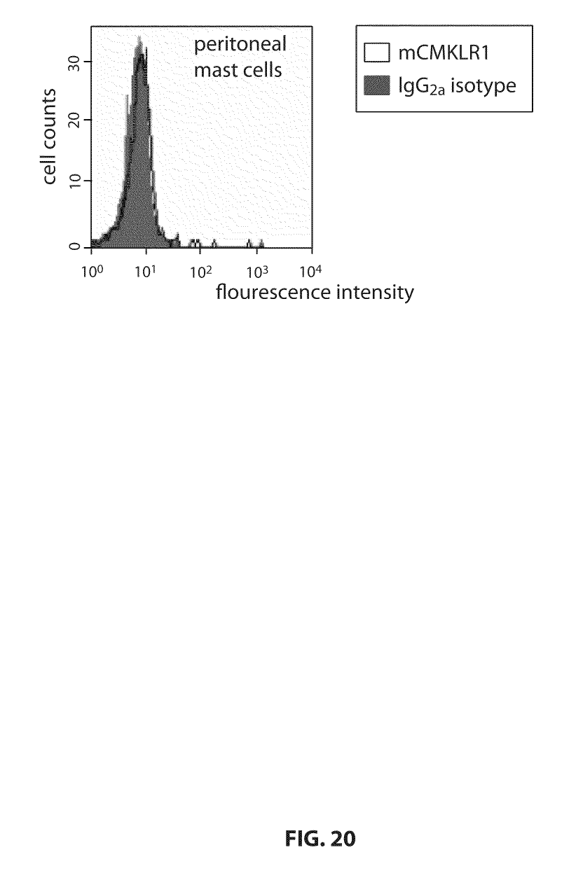

Despite high affinity binding to binding to mCCRL2, chemerin failed to trigger intracellular calcium mobilization in mCCRL2/L1.2 transfectants (FIG. 7, Panel A). Chemerin triggered a robust calcium flux in cells expressing the chemerin signaling receptor mCMKLR1, confirming its activity (FIG. 7, Panel A). mCCRL2-HA/L1.2 transfectants responded to CXCL12 (via endogenous CXCR4), indicating their competence for demonstrating calcium mobilization in this assay (FIG. 7, Panel A). Furthermore, although it was reported that CCL2 triggered intracellular calcium mobilization in CCRL2/HEK293 transfectants, in our experiments neither CCL2 nor chemerin functioned as agonists for CCRL2 in the HEK293 background, either alone or in combination (FIG. 18).

Since GPCR function can require cell type-specific cofactors, we wanted to determine whether CCRL2 could mediate chemerin signaling when expressed physiologically on mouse peritoneal mast cells. Chemerin did not trigger intracellular calcium mobilization in freshly isolated mouse peritoneal mast cells, although these cells responded to ATP (via purinoreceptors (Bulanova, E., V. Budagian, Z. Orinska, M. Hein, F. Petersen, L. Thon, D. Adam, and S. Bulfone-Paus. 2005. Extracellular ATP induces cytokine expression and apoptosis through P2X7 receptor in murine mast cells. J Immunol 174:3880-3890)), indicating their competence in this assay (FIG. 7, Panel B). Furthermore, neither human nor mouse CCRL2-HA/L1.2 transfectants migrated to a range of doses of chemerin in in vitro transwell chemotaxis experiments (FIG. 7, Panel C). Freshly isolated mouse peritoneal mast cells also failed to migrate to chemerin (FIG. 7, Panel D). In comparison, chemerin triggered a robust, dose dependent migratory response in mCMKLR1-HA/L1.2 cells (FIG. 7, Panel C). Mouse and human CCRL2/L1.2 cells migrated to CXCL12 and CCL19 (via endogenously expressed CXCR4 and CCR7, respectively), and primary mouse peritoneal mast cells migrated to stem cell factor (SCF), indicating their ability to demonstrate chemotaxis is this assay (FIG. 7, Panels C-D Furthermore, CCL2 and chemerin did not synergize with each other to induce a functional migratory response in mCCRL2/L1.2 transfectants in in vitro transwell migration assays.

### Example 6

### CCRL2 does not Internalize Chemerin

Our data place CCRL2 in a class of ‘atypical’ receptors that include D6, DARC, and CCX-CKR, all of which bind to chemoattractants but do not support classical ligand-driven signal transduction (Comerford, I., W. Litchfield, Y. Harata-Lee, R. J. Nibbs, and S. R. McColl. 2007. Regulation of chemotactic networks by ‘atypical’ receptors. Bioessays 29:237-247). These other receptors have recently been termed professional “chemokine interceptors” because they internalize and either degrade and/or transcytose chemokines (reviewed in (Mantovani, A., R. Bonecchi, and M. Locati. 2006. Tuning inflammation and immunity by chemokine sequestration: decoys and more. Nat Rev Immunol 6:907-918; Comerford, I., W. Litchfield, Y. Harata-Lee, R. J. Nibbs, and S. R. McColl. 2007. Regulation of chemotactic networks by ‘atypical’ receptors. Bioessays 29:237-247; Haraldsen, G., and A. Rot. 2006. Coy decoy with a new ploy: interceptor controls the levels of homeostatic chemokines. Eur J Immunol 36:1659-1661)). To ask whether CCRL2 might have interceptor activity, we assessed the internalization of CCRL2, and of CMKLR1 for comparison, in response to ligand binding. mCMKLR1-HA internalized rapidly (within 15 min) in response to 100 nM chemerin; and this internalization was inhibited by incubation on ice and in the presence of sodium azide (FIG. 8, Panel A), confirming that the effect is an active process (not due to chemerin-mediated displacement of the anti-HA mAb). In contrast, under the same conditions, CCRL2 failed to internalize: neither mouse nor human CCRL2, expressed on L1.2 transfectants, underwent ligand-induced internalization (FIG. 8, Panel A). Even prolonged incubation with chemerin (2 h at 37° C.) failed to significantly reduce surface receptor levels.

We also asked whether CCRL2 might undergo constitutive, ligand-independent endocytosis, as has been observed with D6 (Weber, M., E. Blair, C. V. Simpson, M. O'Hara, P. E. Blackburn, A. Rot, G. J. Graham, and R. J. Nibbs. 2004. The chemokine receptor D6 constitutively traffics to and from the cell surface to internalize and degrade chemokines. Mol Biol Cell 15:2492-2508). Cell surface mCCRL2-HA and mCMKLR1-HA were stained with primary anti-HA mAb on ice, washed, and then shifted to 37° C. for various times to permit receptor internalization. The cells were then stained with a secondary antibody to detect remaining surface anti-HA mAb. At the 15-min time point, neither mCCRL2 nor mCMKLR1 had undergone substantial ligand-independent endocytosis, similar to CCX-CKR (Comerford, I., S. Milasta, V. Morrow, G. Milligan, and R. Nibbs. 2006. The chemokine receptor CCX-CKR mediates effective scavenging of CCL19 in vitro. Eur J Immunol 36:1904-1916) and in contrast to D6 (Weber, M., E. Blair, C. V. Simpson, M. O'Hara, P. E. Blackburn, A. Rot, G. J. Graham, and R. J. Nibbs. 2004. The chemokine receptor D6 constitutively traffics to and from the cell surface to internalize and degrade chemokines. Mol Biol Cell 15:2492-2508). By 60 min there was a noticeable reduction in staining intensity for both human and mouse receptors, suggesting either low level constitutive endocytosis, receptor turnover, and/or antibody release (FIG. 8, Panel B).

Given that chemerin does not trigger mCCRL2 internalization, it is unlikely that chemerin itself is internalized in substantial amounts in CCRL2+ cells (in the absence of CMKLR1). To confirm this directly, we loaded mCCRL2-HA/L1.2 cells with His₈-tagged serum form chemerin and anti-His₆ PE on ice, and then shifted the cells to 37° C. to permit internalization. At the indicated time points, the cells were washed with either PBS or a high salt acid wash buffer that strips bound chemerin from the surface of the cell (see the zero time point in FIG. 8, Panel C). In contrast to D6 and CCX-CKR, where >70% of cell-associated CCL3 (Weber, M., E. Blair, C. V. Simpson, M. O'Hara, P. E. Blackburn, A. Rot, G. J. Graham, and R. J. Nibbs. 2004. The chemokine receptor D6 constitutively traffics to and from the cell surface to internalize and degrade chemokines. Mol Biol Cell 15:2492-2508) and 100% of cell-associated CCL19 (Comerford, I., S. Milasta, V. Morrow, G. Milligan, and R. Nibbs. 2006. The chemokine receptor CCX-CKR mediates effective scavenging of CCL19 in vitro. Eur J Immunol 36:1904-1916) became resistant to acid wash within 5 min, respectively, there was essentially no acid resistant cell-associated chemerin at the 5 min time point, and very little at the 60 min time point (FIG. 8, Panel C). On the other hand, there was a time-dependent increase in surface bound chemerin (“PBS wash” in FIG. 8, Panel C). Freshly isolated peritoneal mouse mast cells also did not internalize chemerin (“acid wash” in FIG. 8, Panel D). In contrast to mCCRL2/L1.2 transfectants, however, at the 60 min time point there was a considerable reduction in surface bound chemerin (“PBS wash” in FIG. 8, Panel D), suggesting either eventual extracellular degradation or chemerin release. Furthermore, mCCRL2-HA/L1.2 transfectants efficiently bound chemerin from dilute aqueous solutions (FIG. 8, Panel E). Thus, it appears that CCRL2 binds and indeed concentrates chemerin on the cell surface.

Finally, we wanted to assess whether chemerin sequestered by mCCRL2⁺ cells could trigger a response in mCMKLR1⁺ cells. We loaded empty vector pcDNA3 or mCCRL2-HA L1.2 cells with chemerin, washed with PBS, added the loaded cells to mCMKLR1/L1.2 responder cells labeled with a calcium sensitive dye, and assessed intracellular calcium mobilization. Chemerin-loaded mCCRL2-HA/L1.2 cells, but not pcDNA3/L1.2 cells, triggered calcium flux in the responder cells (FIG. 8, Panel F). Thus, CCRL2 can concentrate bioactive chemerin, which then is available for interaction with CMKLR1 on adjacent cells.

### Example 7

### CCRL2 is Upregulated in Bends Endothelial Cells and Binds Chemerin

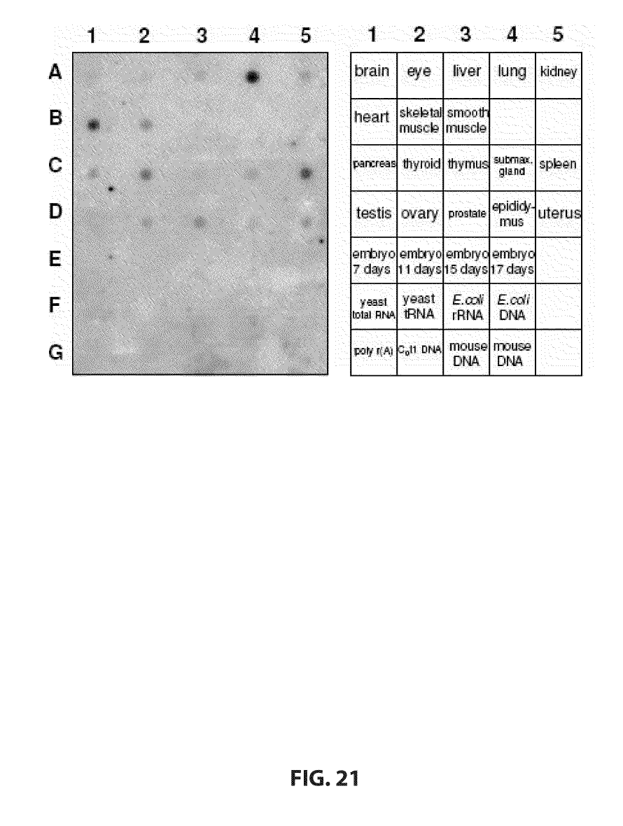

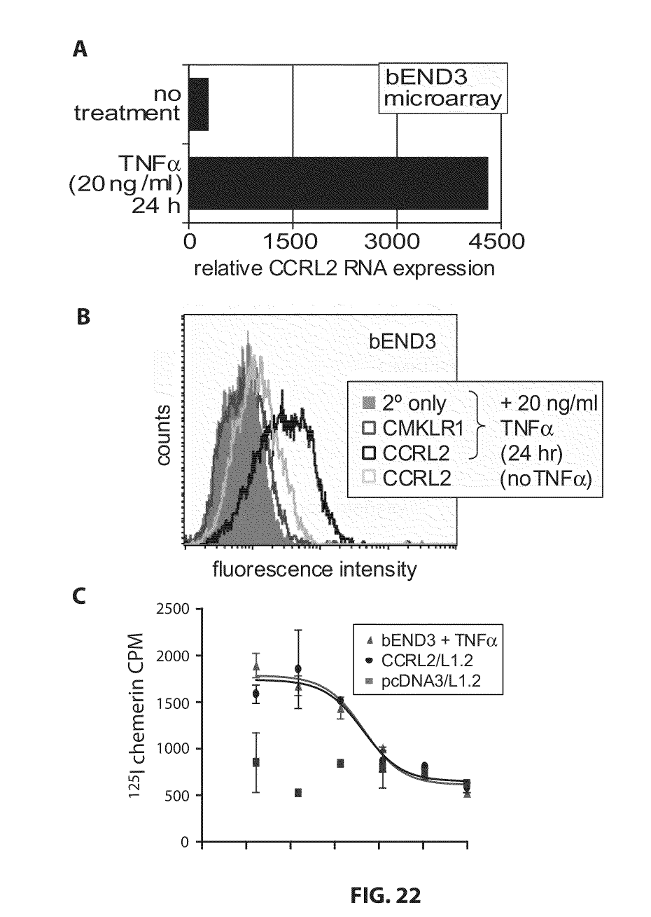

Lande et al. showed that chemerin is associated endothelial cells of inflamed blood vessels in the meninges and white matter lesions of patients with MS (Lande, R., V. Gafa, B. Serafini, E. Giacomini, A. Visconti, M. E. Remoli, M. Severa, M. Parmentier, G. Ristori, M. Salvetti, F. Aloisi, and E. M. Coccia. 2008. J Neuropathol Exp Neurol 67: 388-401) while Vermi et al. showed that chemerin is associated with endothelial cells of inflamed blood vessels in skin lesions of patients with systemic lupus erythematosus (Vermi, W., E. Riboldi, V. Wittamer, F. Gentili, W. Luini, S. Marrelli, A. Vecchi, J. D. Franssen, D. Communi, L. Massardi, M. Sironi, A. Mantovani, M. Parmentier, F. Facchetti, and S. Sozzani. 2005. J Exp Med 201: 509-515). Taken together with our recently published data and our hypothesis that CCRL2 can serve to concentrate chemerin on the cell surface, we asked whether endothelial cells treated with pro-inflammatory stimuli could be induced to express CCRL2 and bind chemerin. Using the mouse brain endothelioma cell line bEND3, we show that CCRL2 mRNA is highly upregulated following 24-hour treatment with 20 ng/ml TNFα (FIG. 21, Panel A). We observed the same TNFα-mediated induction of protein expression by mAb staining (FIG. 21, Panel B). Importantly, expression of CCRL2 correlated with chemerin binding, as shown by radiolabeled chemerin binding (FIG. 21, Panel C). The chemerin binding is through CCRL2, as CMKLR1 is not expressed on the treated bEND3 cells (FIG. 21, Panel B). Thus our data supports the hypothesis that endothelial cells upregulate CCRL2 in response to inflammation and bind and deliver chemerin to CMKLR1+ cells.

### Example 8

### CCRL2 as a Novel Chemerin “Delivery” Receptor Expressed by Activated Endothelium

The role of CCRL2 as a non-signaling receptor that binds chemerin and regulates its bioavailability is also tested. The observations that endothelial cells treated with pro-inflammatory stimuli upregulate CCRL2 and bind chemerin are first tested. Previously we showed that CCRL2+ transfectants and mast cells do not internalize bound chemerin—we propose to extend these experiments to CCRL2+ endothelial cells, as receptor function may vary depending on cell type. Next we propose to characterize the stability of chemerin: CCRL2 interactions and characterize its dissociation kinetics. We will next investigate the role of CCRL2 as a chemerin delivery receptor for CMKLR1. Finally we will test the hypothesis that CCRL2 concentrates chemerin by investigating the co-localization of CCRL2 and chemerin in vivo. Our overall hypothesis is that CCRL2 serves to regulate the bioavailability of chemerin in vivo to fine-tune immune responses mediated via signaling receptor CMKLR1. In addition, it is hypothesized that endothelial cell-expressed CCRL2 may bind and present chemerin to circulating CMKLR1+ cells to control their adhesion and recruitment.

Characterizing the Expression of CCRL2 on Endothelial Cells.

Hypothesis: We will test the hypothesis that CCRL2 is upregulated by vascular endothelial cells in response to proinflammatory stimuli, and that its expression is associated with chemerin protein accumulation.

Rationale: Lande et al. showed that chemerin is associated endothelial cells of inflamed blood vessels in the meninges and white matter lesions of patients with MS (Lande, R., V. Gafa, B. Serafini, E. Giacomini, A. Visconti, M. E. Remoli, M. Severa, M. Parmentier, G. Ristori, M. Salvetti, F. Aloisi, and E. M. Coccia. 2008. J Neuropathol Exp Neurol 67: 388-401)), while Vermi et al. showed that chemerin is associated with endothelial cells of inflamed blood vessels in skin lesions of patients with systemic lupus erythematosus (Vermi, W., E. Riboldi, V. Wittamer, F. Gentili, W. Luini, S. Marrelli, A. Vecchi, J. D. Franssen, D. Communi, L. Massardi, M. Sironi, A. Mantovani, M. Parmentier, F. Facchetti, and S. Sozzani. 2005. J Exp Med 201: 509-515). Taken together with our recently published data and our hypothesis that CCRL2 can serve to concentrate chemerin on the cell surface (Zabel, B. A., S, Nakae, L. Zuniga, J. Y. Kim, T. Ohyama, C. Alt, J. Pan, H. Suto, D. Soler, S. J. Allen, T. M. Handel, C. H. Song, S. J. Galli, and E. C. Butcher. 2008. J Exp Med 205: 2207-2220), we propose examine endothelial cells treated with pro-inflammatory stimuli for CCRL2 expression and chemerin binding. In preliminary studies using the mouse brain endothelioma cell line bEND3, we show that CCRL2 mRNA and protein is upregulated following 24-hour treatment with 20 ng/ml TNFα, and that the treated cells bind chemerin. Thus our preliminary data supports the hypothesis that endothelial cells upregulate CCRL2 in response to inflammation and may bind and deliver chemerin to CMKLR1+ cells.

Experiments and underlying principle: We will confirm our preliminary bEND3 data, and extend it to include treatments with LPS, IFNγ, poly:IC (all shown to upregulate CCRL2 on primary mouse macrophages) and CpG (which had no effect) (24). We will also investigate HUVEC (primary human umbilical vein endothelial cells) for induction of CCRL2 mRNA as well as chemerin binding.

Previously we showed that CCRL2+ transfectants and mast cells do not internalize bound chemerin. As receptor function may vary depending on cell type, we propose to extend these experiments to CCRL2+ endothelial cells. We will load TNFα-treated bEND3 with His₈-tagged serum form chemerin and anti-His₆ PE on ice, and then shift the cells to 37° C. to permit internalization. At various time points, we will wash the cells with either PBS or a high salt acid wash buffer that strips bound chemerin from the surface of the cell. Any remaining fluorescence signal in the acid washed cells would therefore be due to internalization of the labeled ligand.

To evaluate CCRL2 induction on primary vascular endothelial cells, we will inject WT mice with 25 μg/kg TNFα, a dose known to cause upregulation of known endothelial adhesion molecules (Connor, E. M., M. J. Eppihimer, Z. Morise, D. N. Granger, and M. B. Grisham. 1999. J Leukoc Biol 65: 349-355). At various timepoints post TNFα injection, we will inject our FITC-conjugated α-mCCRL2, α-MAdCAM (positive control) or rIgG₂ₐ (negative control) i.v., and then 10 minutes later euthanize the mice. We will isolate various lymph nodes and Peyer's patches, make whole tissue “squashes”, and look for CCRL2 staining on blood vessels. If indicated, alternative stimuli such as LPS may be employed as well.

We will determine if chemerin, identified by i.v. injection of anti-mouse chemerin mAb (R&D Systems) custom labeled with PE, is present on inflamed endothelial cells as reported by Lande et al. (Lande, R., V. Gafa, B. Serafini, E. Giacomini, A. Visconti, M. E. Remoli, M. Severa, M. Parmentier, G. Ristori, M. Salvetti, F. Aloisi, and E. M. Coccia. 2008. J Neuropathol Exp Neurol 67: 388-401) and Vermi et al. (Vermi, W., E. Riboldi, V. Wittamer, F. Gentili, W. Luini, S. Marrelli, A. Vecchi, J. D. Franssen, D. Communi, L. Massardi, M. Sironi, A. Mantovani, M. Parmentier, F. Facchetti, and S. Sozzani. 2005. J Exp Med 201: 509-515), and will test the hypothesis that CCRL2 and chemerin co-localize, consistent with CCRL2 binding and presentation of chemerin to circulating cells.

Anticipated results and discussion: If we observe chemerin internalization by the bEND3 cells, we will follow up to see if chemerin causes intracellular calcium mobilization, and measure other intracellular signaling pathways. If we detect chemerin binding on the treated HUVECs, we will need to assess the cells for CMKLR1 expression, as that may confound the interpretation of the results.

Based on our in vitro studies, we anticipate that TNFα will upregulate CCRL2 on vascular endothelial cells in vivo as well. We hypothesize that venular endothelium, most responsive to inflammatory signals and involved in leukocyte trafficking will be affected; but our analyses will assess arterial and capillary endothelial cells as well. For the in vivo injection experiments, we will use CCRL2 KO mice as a negative control for the 2E3 staining. We will also assess LPS or IFNγ as an alternative to TNFα to stimulate CCRL2 expression. As an alternative to the in vivo injection of FITC-conjugated α-mCCRL2 mAb, we could treat the mice with TNFα, and then, at various time points, euthanize the animals and harvest lymphoid tissue and other organs or tissues of interest (such as brain and skin, as indicated in our rationale section). We can prepare tissue sections and then stain for CCRL2 using standard immunohistochemistry or immunofluorescence techniques. Based on reports of Lande et al. (Lande, R., V. Gafa, B. Serafini, E. Giacomini, A. Visconti, M. E. Remoli, M. Severa, M. Parmentier, G. Ristori, M. Salvetti, F. Aloisi, and E. M. Coccia. 2008. J Neuropathol Exp Neurol 67: 388-401) and Vermi et al. (Vermi, W., E. Riboldi, V. Wittamer, F. Gentili, W. Luini, S. Marrelli, A. Vecchi, J. D. Franssen, D. Communi, L. Massardi, M. Sironi, A. Mantovani, M. Parmentier, F. Facchetti, and S. Sozzani. 2005. J Exp Med 201: 509-515), we expect that chemerin localization on endothelial cells will be observed as well; and that interpretation of the presence or absence of co-localization with CCLR2 will be straightforward. If CCRL2 is co-localized with chemerin on inflamed vascular beds, we will extend the studies to CCLR2 KO mice to confirm the role for CCLR2 in the local chemerin presentation.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of detecting an agent that modulates the activity of CCRL2, the method comprising:
(a) contacting a CCRL2 polypeptide with a candidate agent in the presence of a chemerin polypeptide under conditions, which in the absence of the test agent, permit the binding of the chemerin polypeptide to the CCRL2 polypeptide; and
(b) determining whether the candidate agent is capable of modulating the interaction between said CCRL2 polypeptide and said chemerin polypeptide.

2. A method according to claim 1, wherein the candidate agent is a polypeptide, an antibody or antigen-binding fragment thereof, a lipid, a carbohydrate, a nucleic acid or a chemical compound.

3. A method according to claim 1, wherein step (b) comprises monitoring binding of the CCRL2 polypeptide to the chemerin polypeptide.

4. A method according to claim 3, wherein the binding of the CCRL2 polypeptide to the chemerin polypeptide is monitored using label displacement, surface plasmon resonance, fluorescence resonance energy transfer, fluorescence quenching or fluorescence polarization.

5. A method according to claim 1, wherein the chemerin polypeptide is detectably labelled.

6. A method according to claim 5, wherein the chemerin polypeptide is detectably labelled with a moiety is a radioisotope, a fluorophore, a quencher of fluorescence, an enzyme, an affinity tag or an epitope tag.

7. A method according to claim 1, wherein step (b) comprises monitoring the signalling activity of the CCRL2 polypeptide.

8. A method according to claim 7, wherein the signalling activity is monitored by measurement of guanosine nucleotide binding, GTPase activity, adenylate cyclase activity, cyclic adenosine monophosphate (cAMP), Protein Kinase C activity, phosphatidylinositol breakdown, diacylglycerol, inositol triphosphate, intracellular calcium, MAP kinase activity or reporter gene expression.

9. A method according to claim 1, wherein step (b) comprises monitoring the chemotactic activity of the CCRL2 polypeptide.

10. A method according to claim 1, wherein the CCRL2 polypeptide is expressed on a cell.

11. A method according to claim 10, wherein the cell is a mammalian cell.

12. A method according to claim 10, wherein the cell is a mast cell or macrophage.

13. A method according to claim 1, wherein the CCRL2 polypeptide is present: (a) in or on synthetic liposomes; (b) in or on virus-induced budding membranes; (c) in or on an artificial lipid bilayer; or (d) in a membrane fraction from cells expressing the CCRL2 polypeptide.
